(19) United States
(12) Patent Application Publication
(10) Pub. No.: US 2026/0055429 A1
(43) Pub. Date: Feb. 26, 2026

(54) POLYMER-BASED NANOPLATFORM FOR MRNA DELIVERY TO MULTIPLE CANCER CELL TYPES AND HUMAN INDUCED PLURIPOTENT STEM CELLS

(71) Applicant: University of Washington, Seattle, WA (US)

(72) Inventors: Miqin ZHANG, Seattle, WA (US); Guanyou LIN, Seattle, WA (US); Jianxi HUANG, Seattle, WA (US)

(73) Assignee: University of Washington, Seattle, WA (US)

(21) Appl. No.: 19/105,751

(22) PCT Filed: Aug. 21, 2023
(86) PCT No.: PCT/US2023/030704

§ 371 Date: Feb. 21, 2025

Related U.S. Application Data
(60) Provisional application No. 63/373,325, filed on Aug. 23, 2022.

(51) Int. Cl. C12N 15/88; A61K 9/51; A61K 48/00

(52) U.S. Cl. CPC C12N 15/88; A61K 9/5161; A61K 48/0041

(57) ABSTRACT

A nanoparticle for delivery of mRNA to cell, comprising a core comprising a polyethylenimine polymer having fluorinated groups covalently coupled thereto and with mRNA reversibly associated therewith, and a shell surrounding the core comprising heparin. Ij some embodiments, the nanoparticle comprises a targeting agent associated with the shell, wherein the targeting agent is selected from the group consisting of agents that bind to receptors overexpressed on tumor cells, agents that bind to cell surf ace antigens that are expressed on pluripotent stem cells, agents that bind to cell surface antigens on T cells, and agents that bind to antigens presented by MHO molecules. Pharmaceutical compositions that include the nanoparticle and methods for using the nanoparticle for transfecting cells are provided

### CROSS-REFERENCE TO RELATED APPLICATION

This application claims the benefit of U.S. Patent Application No. 63/373,325, filed Aug. 23, 2022, expressly incorporated herein by reference in its entirety

### STATEMENT OF GOVERNMENT LICENSE RIGHTS

This invention was made with Government support under Grant No. R01EB026890 awarded by the National Institutes of Health. The Government has certain rights in the invention.

### BACKGROUND

Messenger RNA (mRNA), the intermediator between the fixed genetic blueprint (DNA) and the terminal effector (proteins), offers great flexibility and utility as a medicinal agent. The delivery of prophylactic mRNA payloads via nanomaterial-based platforms has showcased its technological prowess in addressing the dire public health challenges the world is facing during recent pandemic years.

Successful mRNA transfection can express virtually any proteins of design in cells and tissues to manipulate cell behaviors and exert prophylactic or therapeutic effects to treat or prevent diseases. The cytosolic mRNA activity which eliminates the need to pass cell's nuclear envelope barrier for transient protein expression and the risk of insertional mutagenesis enables facile and safe transfection. Due to mRNA's labile nature, however, the main challenge of mRNA delivery lies with the development of a reliable carrier offering protection from enzymatic and chemical degradation while ferrying mRNA across biological barriers. The current leading platform for mRNA delivery is lipid nanoparticle (LNP). Nevertheless, LNP-delivered mRNA (LNP-mRNA) formulations often suffer from poor safety profile as they could cause side effects when applied clinically. Recent findings also pointed out that LNP not only encapsulates mRNA but also water pockets which could readily subject mRNA to hydrolysis and jeopardize mRNA's structural integrity unless stored at ultra-cold conditions (−20° C. to −80° C.). The manufacture of LNP-mRNA could be laborious and uneconomical as meticulous mixing of multiple different lipid constituents (usually four) with mRNA followed by ethanol removal necessitates the employment of high precision mixing platform such as rapid microfluidic mixing devices. Therefore, there is a fervor need for a novel class of mRNA delivery platforms alternative to LNP.

Although not as clinically advanced as LNP, cationic polymer-based mRNA delivery platforms have also obtained extensive recognition in research. Different from LNP, a cationic polymer can be simultaneously equipped with multiple functional moieties so that only the multifunctional polymer is needed to complex with mRNA, making the production of polymeric mRNA polyplex more facile and economical than that of LNP-mRNA lipoplex. Due to their larger-than-lipid molecular weight and abundance in positive charge, cationic polymers can form more robust and stable complexes with mRNA which can better protect mRNA from degradation than lipids via multivalent electrostatic condensation. Among innumerous types of cationic polymers, only polyethyleneimine (PEI) is widely applied to deliver mRNA due to its capability of mRNA condensation and endosomal escape. To circumvent PEI's non-biodegradability and cytotoxicity issues, low molecular weight branched PEI-based delivery platforms have been developed and showed great efficacies in delivering mRNA for vaccination against HIV and influenza viruses as well as treating muscle dystrophy, demonstrating low molecular weight branched PEI's utility and suitability for mRNA delivery applications. However, the mRNA transfection efficiency of these mRNA delivery platforms was either inferior to that of LNP or only compared to that of the toxic high molecular weight PEI, leaving low molecular weight PEI-based mRNA delivery platform's transfection efficiency still in doubt.

Despite the advances of mRNA delivery noted above, a need exists for improved mRNA delivery vehicles that enhance transfection efficiency. The present disclosure seeks to fulfill this need and provide further related advantages.

### SUMMARY

In one aspect, the disclosure provides a nanoparticle for delivery of mRNA to cell, comprising:
(a) a core comprising a polyethylenimine polymer having fluorinated groups covalently coupled thereto and with mRNA reversibly associated therewith; and
(b) a shell surrounding the core comprising heparin.

The nanoparticle can include one or more different mRNAs.

The nanoparticle optionally includes a targeting agent associated with the shell.

Pharmaceutical compositions are also provided that include the nanoparticle described herein and a pharmaceutically acceptable carrier.

In another aspect of the disclosure, a method for making the nanoparticle described herein is provided. In one embodiment, the method comprises:
(a) mixing a solution of mRNA with a polyethylenimine polymer having fluorinated groups covalently coupled thereto to provide a polyethylenimine polymer having fluorinated groups covalently coupled thereto with associated mRNA; and
(b) mixing a solution of heparin with the polyethylenimine polymer having fluorinated groups covalently coupled thereto with associated mRNA to provide a polyethylenimine polymer having fluorinated groups covalently coupled thereto with associated mRNA and heparin.

The disclosure also provides methods for using the nanoparticle described herein.

In certain embodiments, the disclosure provides methods for introducing mRNA into a cell or methods for transfecting a cell. In these methods, the cell is contacted with the nanoparticle described herein.

In certain embodiments, the disclosure provides methods for treating a cancer. In these methods, a cancer cell to be treated is contacted with the nanoparticle described herein, or a therapeutically effective amount of the nanoparticle described herein is administered to a subject in need thereof. In these methods, the mRNA of the nanoparticle is effective for treating the cancer.

In another aspect, the disclosure provides a method for inhibiting cancer cell growth. In certain embodiments, the method comprises contacting a cancer cell with the nanoparticle described herein to introduce one or more pre-selected mRNAs into a cell to induce the expression of one or more pre-determined target proteins in the cell thereby inhibiting cancer cell growth or activating immune cells (e.g., T cells and natural killer (NK) cells) to eradicate cancer cells.

In a further aspect, the disclosure provides a method for modifying the function of a stem cell. In certain embodiments, the method comprises contacting a stem cell with the nanoparticle described herein to introduce one or more pre-selected mRNAs into the cell to induce the expression of one or more pre-determined target proteins in the cell thereby modifying the function of the stem cell.

The present disclosure also provides the use of the nanoparticle described herein for introducing mRNA into a cell, transfecting a cell with mRNA, inhibiting cancer cell growth, treating cancer, or modifying the function of a stem cell.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings.

FIGS. 1A-1C illustrate the synthesis of a representative polymeric mRNA delivery nanoplatform disclosed herein (i.e., PFHA-PEI-mRNA-HP). FIG. 1A illustrates a reaction scheme for conjugating PFHA onto PEI via EDC/NHS coupling chemistry. FIG. 1B illustrates the process of mRNA being condensed by PFHA-PEI. mRNA solution was loaded into a syringe and injected into PFHA-PEI solution at a fixed flow rate (1 mL/s) while the solution is stirred by a rotor tip (500 rpm) for homogeneous mixing. FIG. 1C illustrates the process of embellishing the surface of PFHA-PEI-mRNA with HP. HP solution was loaded into a syringe and injected into PFHA-PEI-mRNA solution at a slow flow rate (0.5 mL/s) while the solution is stirred by a rotor tip (500 rpm) for homogeneous mixing.

FIGS. 2A-2H present physicochemical characterization of a representative polymeric mRNA delivery nanoplatform disclosed herein (i.e., PFHA-PEI-mRNA-HP). FIG. 2A illustrates the FTIR spectra of PFHA, PEI and PFHA-PEI. The dashed box marks the region of the addition of characteristic peak patterns from PFHA's spectrum onto PEI's spectrum. The dashed line indicates the presence of the amide bonds formed between PFHA and PEI. FIG. 2B illustrates the X ray photoelectron spectroscopy (XPS) spectrum of PFHA-PEI with peak fitting analysis. Hydrodynamic size (FIG. 2C), polydispersity index (FIG. 2D) and zeta potential (FIG. 2E) measurements of PEI-mRNA, PFHA-PEI-mRNA and PFHA-PEI-mRNA-HP with various HP amounts. For the labels on the x axis of FIGS. 2C-2E, PEI represents PEI-mRNA. 0, 1, 2, 5 correspond to PFHA-PEI-mRNA+0, 1, 2, 5 mg HP/mg mRNA. FIG. 2F illustrates a gel retardation assay of PEI-mRNA, PFHA-PEI-mRNA and PFHA-PEI-mRNA-HP (with different HP amounts) with free mRNA as control. FIG. 2G illustrates serum stability data for PEI-mRNA, PFHA-PEI-mRNA and PFHA-PEI-mRNA-HP with mRNA:HP wt/wt ratio of 1:1. All samples were placed in PBS supplemented with 10% v/v FBS solutions and incubated at 37° C. FIG. 2H show TEM images of PEI-mRNA, PFHA-PEI-mRNA and PFHA-PEI-mRNA-HP (with different HP amounts). The scale bars in different TEM images are different as some aggregates are too large to be captured in whole.

FIGS. 3A-3D depict endosomal escape studies on 3 different cell types at 37° C. and 4° C. at 12-h time points. FIG. 3A shows confocal images of 3 different cell lines treated with PEI-mRNA, PFHA-PEI-mRNA and PFHA-PEI-mRNA-HP. Cy5-labeled mRNA was complexed, added to cell cultures at 2 mg/mL and incubated at 37° C. for 12 hours. Lysotracker Red reagent was then added to cells and incubated for another 1 hours. mRNA-Cy5, Lysotracker, and DAPI nuclear stain are illustrated. Scale bar is 20 mm. Arrows point at the regions where distinguished separation between mRNA signal and Lysotracker signal. FIG. 3B compares signal intensity of intracellular mRNA uptake quantified from the confocal images in FIG. 3A. Statistical analysis was performed by comparing the PEI-mRNA, PFHA-PEI-mRNA and PFHA-PEI-mRNA-HP groups to the untreated group. FIG. 3C shows Pearson colocalization coefficient analysis of the confocal images of the PFHA-PEI-mRNA-HP treated cells in FIG. 3A. FIG. 3D shows confocal images of 3 different cell lines treated with PFHA-PEI-mRNA-HP. The experimental procedure is the same as that from FIG. 3A except that the incubation temperature was at 4° C. mRNA-Cy5, Lysotracker, and DAPI nuclear stain are illustrated. Arrows point at the locations where PFHA-PEI-mRNA-HP nanoparticles are stranded on cell plasma membrane. Scale bar is 10 mm.

FIGS. 4A and 4B compare cell viability test results of a representative polymeric mRNA delivery nanoplatform disclosed herein (i.e., PFHA-PEI-mRNA-HP). FIG. 4A presents Quantitative Alamar Blue cell viability assay results on 4T1, HepG2 and MMC. Each cell type was treated by PEI-mRNA, PFHA-PEI-mRNA, PFHA-PEI-mRNA-HP and Lipofectamine 2000-mRNA at 0,5, 1, 2 and 3 mg/mL for 24 hours. The untreated cells' viability was normalized to 100% for all cell lines. Statistical analysis was performed to determine if the difference between the data points from the Lipo-mRNA-treated cells and the data points from other treated cells were significant. FIG. 4B shows representative bright field images of the cells either untreated or treated with PFHA-PEI-mRNA-HP at 2 mg/mL mRNA concentration. Scale bar is 50 mm.

FIGS. 5A-5C compare transfection results for three different cancer cell lines. FIG. 5A shows transfection images of PEI-mRNA, PFHA-PEI-mRNA and PFHA-PEI-mRNA-HP with Lipofectamine 2000-mRNA as positive control on 4T1, HepG2 and MMC cells. Scale bar is 100 mm. FIG. 5B shows quantitative analysis of the transfection results presented in FIG. 5A. Statistical analysis was performed by comparing each of the treatment groups to the positive control lipo2000-mRNA group. FIG. 5C shows flow cytometric quantitative analysis of transfection efficiency of PFHA-PEI-mRNA-HP with Lipofectamine 2000-mRNA as positive control on 3 cancer cell lines.

FIGS. 6A-6C compare transfection results for three additional cancer cell lines. FIG. 6A shows transfection images of PEI-mRNA, PFHA-PEI-mRNA and PFHA-PEI-mRNA-HP with Lipofectamine 2000-mRNA as positive control on C6, SF763 and MCF7 cells. Scale bar is 100 mm. FIG. 6B shows quantitative analysis of the transfection results presented in FIG. 6A. Statistical analysis was performed by comparing each of the treatment groups to the positive control lipo2000-mRNA group. FIG. 6C shows flow cytometric quantitative analysis of transfection efficiency of PFHA-PEI-mRNA-HP with Lipofectamine 2000-mRNA as positive control on the additional 3 cancer cell lines.

FIGS. 7A and 7B compare above 0° C. storage stability test on 4T1 and HepG2 cells. PFHA-PEI-mRNA-HP and Lipofectamine 2000-mRNA were prepared on day 0 and refrigerated at 4° C. throughout the course of study. PFHA-PEI-mRNA-HP and Lipofectamine 2000-mRNA were allowed to equilibrate to room temperature before they were added to 4T1 and HepG2 cell cultures at 2 mg/mL mRNA concentration on day 0, 1, 2, 3, 4, 7 and 15. FIG. 7A shows fluorescent images of transfected cells. The images were collected 24 hours after PFHA-PEI-mRNA-HP and Lipofectamine 2000-mRNA were added on each day. Scale bar is 100 mm. FIG. 7B shows quantification of the fluorescence intensities shown in the images. Fluorescence intensities in each panel were normalized against the intensity at day 0 which was assigned as 100%.

FIGS. 8A and 8B compare therapeutic effects of targeting ligand functionalized PFHA-PEI-IL12+IFNγ mRNA-HP in TNBC M6 tumor-bearing mice. FIG. 8A compares tumor volume measurements of the treated M6 mice and FIG. 8B compares body weight measurements of the treated M6 mice (n=3).

FIG. 9 compares cellular uptake and endosomal escape studies on human induced pluripotent stem cells (hiPSCs) at 4° C. or 37° C. Cy5-labeled mRNA was complexed with PFHA-PEI-LHP and added to cell cultures at 2 mg/mL mRNA dosage and incubated for 12 hours at either 4° C. or 37° C. before adding Lysotracker Red reagent and incubated for another 1 hours at their corresponding temperatures. mRNA-Cy5 signals, Lysotracker, and DAPI nuclear stain are shown with bright field images shown in background. Scale bar is 20 mm. In the 4° C. PFHA-PEI-mRNA-LHP images, arrows point at the locations where PFHA-PEI-mRNA-LHP nanoparticles are stranded on cell plasma membrane. In the 37° C. PFHA-PEI-mRNA-LHP images, arrows point at PFHA-PEI-mRNA-LHP nanoparticles which are deeply internalized and separated from endo-lysosomes.

FIGS. 10A and 10B compare endosomal escape studies on hiPSCs at 37° C. at 12-h time points (3 h and 13 h). Confocal images of hiPSCs treated with PEI, PFHA-PEI, and PFHA-PEI-LHP are shown in FIG. 10A. Cy5-labeled mRNA was complexed with and added to cell cultures at 2 mg/mL and incubated at 37° C. for 12 hours or before adding Lysotracker Red reagent and incubated for another 1 hour. mRNA-Cy5 signal, Lysotracker, and DAPI nuclear stain are shown. Scale bar is 20 mm. Arrows point at the regions where distinguished separation between mRNA signal and Lysotracker signal. One representative region in the PFHA-PEI-mRNA-LHP image from each cell line is magnified into a separate image (the most right column). The dashed lines in the magnified images correspond to the distance where fluorescence intensity line profiles were analyzed. Signal intensity line profile analysis over the distance indicated by the dashed lines in the magnified images from the PFHA-PEI-mRNA-LHP panel are shown in FIG. 10B.

FIG. 11 compares transfection results on hiPSCs. Transfection images of PEI-mRNA, PFHA-PEI-mRNA, and PFHA-PEI-mRNA-LHP with Lipofectamine 2000-mRNA as positive control on hiPSCs. Scale bar is 100 mm.

FIG. 12 compares cell viability test results on hiPSCs. PEI-mRNA, PFHA-PEI-mRNA, PFHA-PEI-mRNA-LHP and Lipofectamine 2000-mRNA were applied to hiPSCs at 0.5, 1, 2 and 3 mg/mL for 24 hours before Alamer blue assay was performed. The untreated cells' viability was normalized to 100%. Statistical analysis was performed to determine if the difference between the data points from the Lipo-mRNA-treated cells and the data points from the PFHA-PEI-mRNA-LHP treated cells were significant.

FIG. 13 compares the HPLC analysis of PEI, PFHA, and PFHA-PEI. The mobile phase consisted of two solvents: A 85% acetonitrile and B 15% DI water. The separation was performed using a linear gradient of A-B (v/v). The flow rate was maintained at 0.2 mL/min. Absorbance was monitored at 220 nm. The retention time for PEI, PFHA and PFHA-PEI were 36 mins, 18 mins and 23 mins respectively.

FIG. 14 compares Raman spectra of PEI, PFHA and PFHA-PEI. Vertical dashed lines indicate the common peaks shared by both PFHA and PFHA-PEI and vertical dashed lines indicate the common peaks shared by both PEI and PFHA-PEI. The left and right square boxes on the PFHA-PEI spectrum indicate the presence of amide III and amide I, respectively.

FIG. 15 illustrates the 19F NMR spectrum of PFHA-PEI. Trifluoroacetic acid (TFA) was used as reference. Peaks corresponding to fluorine atoms in the spectrum are highlighted. The peaks at −76 ppm (area integral of 43.813) and −82 ppm (area integral of 37.8085) correspond to TFA and PFHA. Quantitative analysis results indicate that the molar ratio between PFHA and PEI in PFHA-PEI is 5.479:1.

FIG. 16 compares mRNA encapsulation efficiency of PFHA-PEI-mRNA and PFHA-PEI-mRNA-HP. The mRNA content in the supernatant of each group was normalized against the pure mRNA (100%) positive control group. PFHA-PEI-HP without mRNA served as the negative control. The mRNA encapsulation efficiency was calculated by 100%−mRNA % in the supernatant.

### DETAILED DESCRIPTION

Fluorine has been widely utilized in medicinal industry to modify drugs' molecular structures for better pharmacokinetic and therapeutic outcomes and imaging application purposes. Recent discoveries found that fluorination could substantially improve the gene delivery efficacy of cationic polymers. This phenomenon could be mostly attributed to the unique properties of fluorine. Being simultaneously hydrophobic and lipophobic, fluorocarbon chain exhibits biphasic separation at aqueous-organic interface and energy-favorable self-assembly. These unique characteristics of fluorocarbon empower fluorinated cationic polyplexes with structural compactness and stability as a result of its propensity to self-assemble, biocompatibility due to its inertness and low surface energy, and ability to smoothly traverse biological barriers such as the plasma and endo-lysosomal membranes due to its biphasic separation property. Fluorinated cationic polyplexes have been reported to have high efficiency in delivering DNA, siRNA, and proteins, but none has been reported for mRNA delivery.

A common dilemma for polymeric gene delivery platforms is that the high density of cationic charges necessary for effective nucleic acid condensation also poses issues of toxicity, insufficient nucleic acid release and serum protein adsorption. A promising solution for these problems is embellishing cationic polyplexes with polyanions. Adding polyanions not only improves complex's biocompatibility and serum stability by partially shielding complex's positive surface charge but also helps tune the binding tightness between cationic polymers and nucleic acids so that a subtle packing-unpacking balance can be achieved for efficient nucleic acid release. As a biocompatible polysaccharide with high anionic charge density, heparin (HP) has been repeatedly reported to significantly improve various types of cationic polyplexes' biocompatibility, nucleic acid release profile and transfection efficiency when incorporated. Moreover, HP can bind to the fibroblast growth factor receptor (FGFR) whose aberrant and amplified expression is responsible for the oncogenesis of various types of cancers, making heparin a targeting moiety for preferential uptake in cancer cells. Although the polyanion embellishment strategy has been proven effective for DNA and RNAi delivery, whether the same strategy would display similar enhancement effect on mRNA delivery platforms remained largely unexplored, if not completely unknown.

To this end, the present disclosure provides a polymeric mRNA delivery platform (termed “PFHA-PEI-mRNA-HP”) combining the merits of low molecular weight branched PEI, fluorination, and heparin embellishment, and demonstrates its utility in transfection of multiple cell types (e.g., cancer and stem cells) for the first time. Branched PEI with 2 kDa molecular weight, perfluoroheptanoic acid (PFHA) as the fluorocarbon moiety, and low molecular weight (1.8 kDa-7.5 kDa) heparin (HP) were selected as the constituents of this mRNA delivery platform. PFHA-PEI-mRNA-HP possessed a sub-hundred nm size, spherical shape and sufficient positive surface charge which are conducive for effective mRNA delivery. Because the capability of achieving successful gene delivery in cancer cells is useful in improving the therapeutic outcomes of cancer treatments, PFHA-PEI-mRNA-HP was applied to different types of cancer cells to test its in vitro mRNA delivery utility. Breast and liver cancer cells were chosen as the target cells as they are major types of cancers inflicting large number of deaths worldwide (over 1.5 million in 2020). Brain cancer cells were also tested because brain cancer is one the deadliest cancer types with a 5-year survival rate below 5% even though it is not as prevalent as breast and liver cancers. Notably, PFHA-PEI-mRNA-HP was able to achieve ultra-high mRNA transfection efficiency (>90%) on certain types of breast cancer cells, brain cancer cells and liver cancer cells. Mechanistic studies revealed that PFHA contributed to the desired compact size and spherical shape of PFHA-PEI-mRNA when PEI alone could not effectively condense mRNA. The biphasic separation properties of PFHA substantially boosted the cell uptake compared to PEI-mRNA and endowed PFHA-PEI-mRNA with the capability to effectively escape from endo-lysosomes. The addition of HP to PFHA-PEI-mRNA further greatly enhanced PFHA-PEI-mRNA-HP's cellular uptake and transfection performance compared to PFHA-PEI-mRNA. Moreover, PFHA-PEI-mRNA-HP exhibited a stability greater than Lipofectamine 2000-mRNA when stored at 4° C. for 15 days. The phenomenal mRNA delivery performance and storage stability demonstrated that PFHA-PEI-mRNA-HP can be a highly efficient and robust mRNA delivery platform for anti-cancer gene therapy.

The prophylactic and therapeutic utilities of mRNA are being actively studied and applied to address public health challenges including the pandemic caused by coronavirus and cancer. Because mRNA is labile in nature, reliable delivery platforms are used for successful mRNA-based therapies. Lipid Nanoparticle (LNP) is currently the most clinically advanced mRNA delivery platform but suffers from limitations in safety profiles, storage restrictions and costly manufacture processes. Alternatively, polymeric mRNA delivery platforms have shown great promise due to their structural versatility, robustness, and transfection efficiency. The present disclosure provides a polymeric mRNA delivery nanoplatform (termed “PFHA-PEI-mRNA-HP”). Simultaneous fluorination and heparinization of low molecular weight PEI-based mRNA complex significantly improved its physicochemical properties, cellular uptake and endosomal escape capability, biocompatibility, and thus significantly increased the transfection efficiency. As a result, this polymeric mRNA delivery nanoplatform was able to achieve ultra-high transfection efficiency (>90%) across multiple types of cancer cells compared to that achieved by LNP delivery reagent, Lipofectamine 2000. The polymeric mRNA delivery nanoplatform also exhibited greater stability than Lipofectamine 2000 while being stored at above 0° C. for 15 days. Moreover, the polymeric mRNA delivery nanoplatform also showed reliable serum stability and innocuous toxicity profile on multiple types of cancer cells. These results demonstrated the polymeric mRNA delivery nanoplatform's ability to serve as a highly efficient and reliable mRNA delivery platform for gene therapies.

In one aspect, the present disclosure provides compositions for delivery of mRNA to cell. The composition for delivery of mRNA is a polymeric mRNA delivery nanoplatform.

In certain embodiments, the polymeric mRNA delivery nanoplatform is a nanoparticle. In certain of these embodiments, the nanoparticle comprises:
(a) a core comprising a polyethylenimine polymer having fluorinated groups covalently coupled thereto and with mRNA reversibly associated therewith; and
(b) a shell surrounding the core comprising heparin.

The nanoparticle can include one or more different mRNAs.

The nanoparticle is a polyethylenimine polymer complex for delivery of mRNA to cells that comprises a polyethylenimine polymer having fluorinated groups covalently coupled thereto and with mRNA reversible associated therewith and with heparin associated therewith.

In certain embodiments, the nanoparticle further comprises a targeting agent associated with the shell. The targeting agent assists in directing the nanoparticle to the cell of interest. Suitable targeting agents include agents that bind to receptors overexpressed on tumor cells, agents that bind to cell surface antigens that are expressed on pluripotent stem cells, agents that bind to cell surface antigens on T cells, and agents that bind to antigens presented by MHC molecules.

Tumor target agents are molecules that specifically bind to receptors overexpressed on tumor cells. Examples include antibodies against proteins or markers overexpressed on the surface of cancer cells, peptides that bind to tumor-specific receptors, folate receptor ligands that target highly expressed folate receptors on tumor cells, epidermal growth factor receptor (EGFR) ligands that bind to EGFR often overexpressed in cancer cells, and transferrin receptor (TfR) ligands that target receptors frequently overexpressed on the surface of cancer cells. Useful target agents for brain tumor are chlorotoxin (also known as TM-601), folate, EGFR ligand, ligands for αvβ3 Integrin, and TFR ligand. Useful target agents for breast cancer are anti-neu antibody (antibody for human epidermal growth factor receptor 2), and endoglin-binding peptide (EBP). Useful target agents for liver cancer are glypican 3, anti-CD133 antibody, EGFR ligands, vascular endothelial growth (VEGF) ligand. Useful target agents for human pluripotent stem cells are antibodies against SSEA-3 and SSEA-4, October 4, and Nanog can be used to target hiPSCs. Stage-Specific Embryonic Antigens (SSEA): SSEA-3 and SSEA-4 are cell surface antigens that are expressed on pluripotent stem cells, including hiPSCs. Octamer-Binding Transcription Factor 4 (Oct-4): Oct-4 is a transcription factor involved in maintaining pluripotency. Nanog: Nanog is another transcription factor for pluripotency. Nanog-targeting antibodies can be utilized to study and characterize hiPSCs. Useful target agents for T cells are antibodies against MHC, TCR, CD28; and CD80 and CD86 molecules. Major histocompatibility complex (MHC) molecules present antigens to T cells, enabling them to recognize and respond to infected or abnormal cells. T Cell Receptor (TCR) ligands are specific antigens presented by MHC molecules, initiating the T cell activation process. Co-stimulatory molecules like CD80 and CD86 on antigen-presenting cells interact with CD28 on T cells, providing co-stimulatory signals for T cell activation. The targeting agent can be associated with the nanoparticle by attaching the targeting agent to the nanoparticle shell.

The nanoparticle described herein includes a polyethylenimine polymer. As used herein the term “polyethylenimine polymer” refers to a branched polyethylenimine polymer. The polyethylenimine polymer includes primary, secondary, and tertiary amine groups.

The nanoparticle described herein includes mRNA that is deliverable to a cell. The mRNA is reversibly associated with the nanoparticle described herein. As used herein, the term “reversibly associated” refers to the delivery (i.e., release) of the mRNA from the complex/nanoparticle once the complex/nanoparticle arrives at the site of mRNA delivery (e.g., a targeted site such as a lysosome where the lysosome's environment results in release of at least a portion of the mRNA molecules from the complex/nanoparticle). The complex/nanoparticle selectively delivers the mRNA and does not release the mRNA prematurely, such as in the blood stream (circulatory system). The nanoparticle may include and deliver one or more different therapeutic mRNAs.

The nanoparticle described herein includes a polyethylenimine (PEI) polymer having a molecular weight from about 0.8 kDa to about 8 kDa. In certain embodiments, the polyethylenimine polymer has a molecular weight of about 2 kDa.

The nanoparticle described herein includes a polyethylenimine polymer having fluorinated groups covalently coupled thereto. In certain embodiments, each fluorinated group has a molecular weight of about 350 g/mole. In certain embodiments, the fluorinated group includes from about 11 to about 15 fluoro (—F) groups. In certain embodiments, the fluorinated group from about 4 to about 6 difluoromethylene (—CF₂—) groups. Representative fluorinated groups include C₁-C₁₂ perfluoroalkyl groups (i.e., —(CF₂)ₙCF₃, where n is an integer from 1 to about 11. In one embodiment, the fluorinated group is —(CF₂)₅CF₃ (e.g., the perfluoroalkyl group of perfluoroheptanoic acid).

In certain embodiments, the fluorinated group is present in the nanoparticle from about 45 to about 60 weight percent based on the total weight of the polyethylenimine polymer. In certain embodiments, the molar ratio of fluorinated group to PEI polymer to which the fluorinated group is covalently coupled is from about 5 to about 9. On average, each 2 kDa branched PEI molecule has about 7 PFHA molecules covalently conjugated thereto.

The nanoparticle described herein includes mRNA (e.g., one or more different mRNAs). In certain embodiments, the mRNA comprises from about 20 to about 4000 nucleotides. In certain embodiments, the weight ratio of the mRNA to polymer complex is about 15:1. In certain embodiments, the weight ratio of mRNA to heparin in the polyethylenimine polymer complex is from about 1:1 to about 1:5.

In certain of these embodiments, the mass ratio between PFHA-PEI polymer and mRNA is from about 5:1 to about 20:1. In certain embodiments, the mass ratio is about 15:1. In certain embodiments, the mass ratio between LHP and mRNA is from about 0.1:1 to about 1.5:1. In certain of these embodiments, the mass ratio is about 1:1.

The nanoparticle described herein includes heparin. In certain embodiments, the heparin has a molecular weight from about 1.0 kDa to about 30 kDa. In certain of these embodiments, the heparin has a molecular weight from about 1.8 kDa to about 7.5 kDa. In certain embodiments the heparin has a molecular weight of about 5 kDa. In certain embodiment, the heparin is present in the polymer from about 1 to about 8 weight percent based on the total weight of the polymer, or the molar ratio of the heparin to PEI polymer complex is from about 1:10 to about 1:200. In certain embodiments, the mass ratio between heparin and PFHA-PEI polymer is from about 0.1:15 to 1.5:15. In certain of these embodiments, the mass ratio is 1:15.

In certain embodiments, the nanoparticle has a hydrodynamic size from about 80 to about 150.

In certain embodiments, the nanoparticle has a polydispersity index from about 0.1 to about 0.25.

In certain embodiments, the polyethylenimine polymer complex or the nanoparticle has a zeta potential from about 30 to about 40 mV.

In another aspect, the disclosure provides a pharmaceutical composition comprising the nanoparticle as described herein and a pharmaceutically acceptable carrier (e.g., dextrose or saline solution for injection).

In a further aspect, the disclosure provides a method for making a nanoparticle for delivery of mRNA to cell, comprising:
(a) mixing a solution of mRNA with a polyethylenimine polymer having fluorinated groups covalently coupled thereto to provide a polyethylenimine polymer having fluorinated groups covalently coupled thereto with associated mRNA; and
(b) mixing a solution of heparin with the polyethylenimine polymer having fluorinated groups covalently coupled thereto with associated mRNA to provide a polyethylenimine polymer having fluorinated groups covalently coupled thereto with associated mRNA and heparin.

In certain embodiments, mixing the solution of mRNA with the polyethylenimine polymer having fluorinated groups covalently coupled thereto to provide the polyethylenimine polymer having fluorinated groups covalently coupled thereto with associated mRNA comprises adding mRNA to a solution of the polyethylenimine polymer having fluorinated groups covalently coupled thereto.

In certain of these embodiments, mixing the solution of heparin with the polyethylenimine polymer having fluorinated groups covalently coupled thereto with associated mRNA to provide the polyethylenimine polymer having fluorinated groups covalently coupled thereto with associated mRNA and heparin comprises adding heparin to a solution of the polyethylenimine polymer having fluorinated groups covalently coupled thereto with associated mRNA.

In certain embodiments of the above methods, mixing comprises rotor-syringe mixing.

In another aspect of the disclosure, methods are provided for using the nanoparticle described herein.

In certain embodiments, the disclosure provides a method for introducing mRNA (e.g., one or more different therapeutic mRNAs introduced via a single nanoparticle) into a cell, comprising contacting a cell with the nanoparticle described herein.

In other embodiments, the disclosure provides a method for transfecting a cell, comprising contacting a cell to be transfected with the nanoparticle described herein.

In certain embodiments, the disclosure provides a method for treating a cancer, comprising contacting a cancer cell to be treated with the nanoparticle described herein, wherein the mRNA of the nanoparticle is a therapeutic mRNA effective for treating the cancer.

In other embodiments, the disclosure provides a method for treating a cancer in a subject, comprising administering to a subject in need thereof a therapeutically effective amount of the nanoparticle described herein, wherein the mRNA of the nanoparticle is a therapeutic mRNA effective for treating the cancer.

In certain of the above methods, the cell is a cancer cell. Representative cancer cells include brain cancer cells, breast cancer cells, liver cancer cells, ovarian cancer cells, prostate cancer cells, kidney cancer cells, lymphoma cells, melanoma cells, sarcoma cells, and their corresponding cancer stem cells.

In other of the above methods, the cell is a human induced pluripotent stem cell, a T cell, a neural cell, a fibroblast, a muscle cell, a cartilage cell, or a bone cell.

In certain of the above methods, the cancer is a brain cancer, a breast cancer, a liver cancer, an ovarian cancer, a prostate cancer, a kidney cancer, a lymphoma, a melanoma, or a sarcoma.

Introducing mRNA into a cell, also known as transfecting a cell with mRNA, is useful for modifying the function and properties of the cell to provide a transfected cell. Transfection results in changes in cell function and properties because the mRNA introduced into the cell provides instructions for protein synthesis thereby influencing cell function through the creation of proteins that drive essential cellular activities. Described herein are formulations and methods for mRNA transfection, tailored for modification of cancer cells and stem cells. As is well known, there are significant differences between cancer cells and stem cells in terms of their cell membrane properties, growth, and function. As is also well known and as described herein, the cellular uptake of mRNA vehicles (e.g., the representative polymeric mRNA delivery nanoplatform disclosed herein, such as PFHA-PEI-mRNA-HP) and therefore the uptake of mRNA differs between cancer stem cells and cancer cells, resulting in distinct transfection efficiencies for each cell type.

In certain embodiments, the disclosure provides a method for inhibiting cancer growth using the polymeric mRNA delivery nanoplatform disclosed herein. As described herein, the cancer cell is transfected with mRNA using the polymeric mRNA delivery nanoplatform resulting in cancer cell death.

Thus, in one embodiment, the disclosure provides a method for inhibiting cancer cell growth comprising contacting a cancer cell with a nanoparticle as described herein to introduce one or more pre-selected therapeutic mRNAs into the cell to induce the expression of one or more pre-determined target proteins in the cell thereby inhibiting cancer cell growth or activating immune cells (e.g., T cells and natural killer (NK) cells) to eradicate cancer cells.

In other embodiments, the disclosure provides a method for modifying the function of a stem cell using the polymeric mRNA delivery nanoplatform disclosed herein. As described herein, the stem cell is transfected with mRNA using the polymeric mRNA delivery nanoplatform resulting in delivery of mRNA within the cell and a modification of the stem cell's function. Such modified stem cells can be used in regenerative medicine.

Thus, in another embodiment, the disclosure provides a method for modifying the function of a stem cell, comprising contacting a stem cell with a nanoparticle as described herein to introduce one or more pre-selected therapeutic mRNAs into the cell to induce the expression of one or more pre-determined target proteins in the cell thereby modifying the function of the stem cell.

In the above methods, the cell is transfected with one or more pre-selected mRNAs that are effective to induce the expression of one or more pre-determined target proteins in the cell. The correlation between the pre-selected mRNA and the pre-determined target protein is known to the skilled person. In practice, the polymeric mRNA delivery nanoplatform disclosed herein includes the pre-selected mRNA (one or more types) and the polymeric mRNA delivery nanoplatform serves as the vehicle for delivering the mRNA within the cell.

The preparation, characterization, evaluation and use of a representative polymeric mRNA delivery nanoplatform, PFHA-PEI-mRNA-HP, is described below.

Design and Synthesis of a Polymeric mRNA Delivery Nanoplatform: PFHA-PEI-mRNA-HP

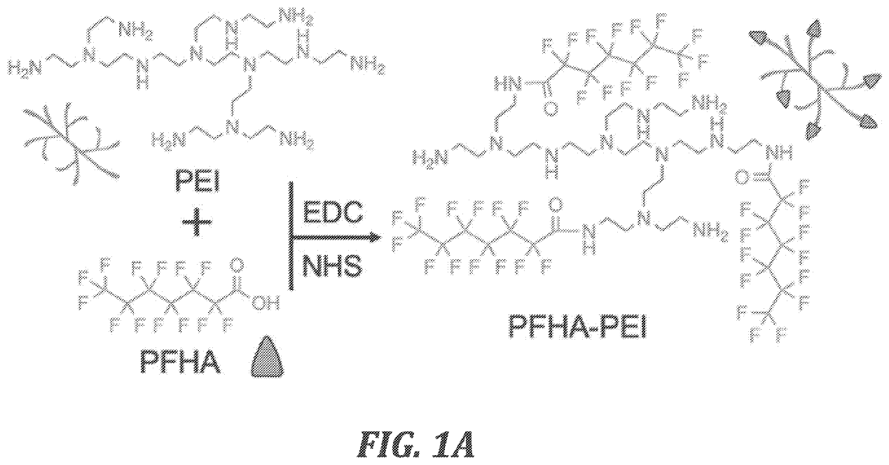

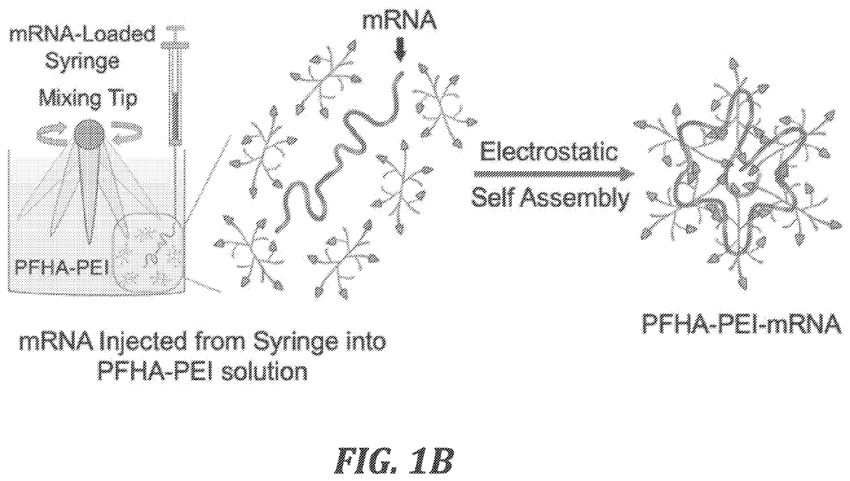

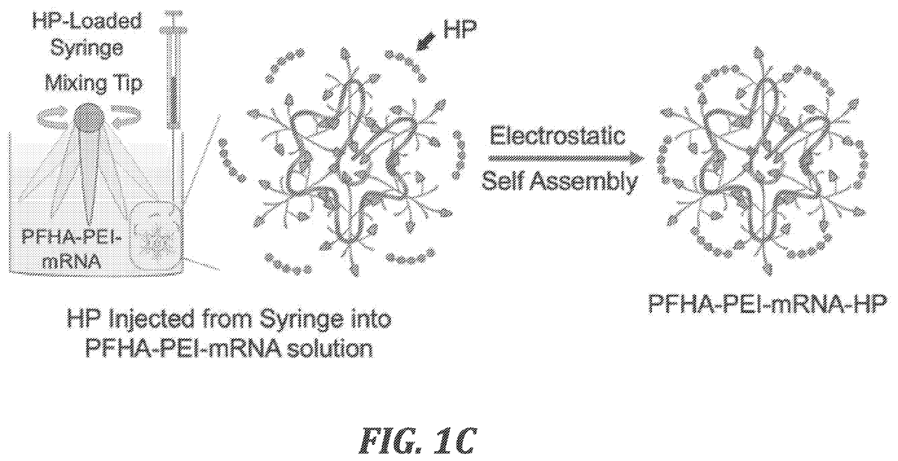

The molecular properties such as molecular weight, polarity and functional groups of each of PFHA-PEI-mRNA-HP's constituents were taken into consideration for selection. PFHA was chosen based on the consideration of appropriate PFHA chain length as PFHA being too long would compromise mRNA complex's aqueous solubility while too short would diminish PFHA's utility in the system. Branched PEI with 2 kDa molecular weight was selected for its relatively strong nucleic acid condensing capability and innocuous toxicity profiles. Given that high molecular weight heparin could compete with mRNA for electrostatic binding and cause large-size aggregation, HP was chosen due to its small size which is beneficial for controlling the size and integrity of mRNA complex. PFHA was conjugated on PEI (branched, MW 2 kD) via EDC/NHS coupling chemistry (FIG. 1A). The PFHA:PEI molar ratio for coupling was set at 7:1 for conjugation as this ratio (i.e., PFHA:PEI/7:1) yielded a useful transfection result.

A rotor-syringe mixing (RSM) platform was set up by combining a microliter syringe-loaded syringe pump, a mechanical rotor equipped with a disposable stirring head and a lifting sample tube holder into a solution mixing system to assemble PFHA-PEI, mRNA and HP into PFHA-PEI-mRNA-HP nanoparticles. With precise control over the stirring speed and injection flow rate, the RSM platform ensures consistent mixing efficiency and complexing outcomes when making mRNA complex. The core of nanoparticle is composed of mRNA condensed by PFHA-PEI to render structural compactness for mRNA protection. PFHA-PEI-mRNA complex was first formed by slowly injecting mRNA solution at 1 mL/s into PFHA-PEI solution which was being stirred at 500 RPM by the RSM platform (FIG. 1B). Injecting mRNA into PFHA-PEI solution instead of the other way around ensures that each individual mRNA molecule can be fully covered and condensed upon contact with PFHA-PEI. The PFHA-PEI: mRNA wt/wt ratio was set at 15:1 for useful physicochemical properties and transfection compared to other ratios.

The surface of the PFHA-PEI-mRNA core was then decorated with HP to form an outer shell layer for tuning the binding tightness of mRNA in the core and in turn facilitating the intracellular delivery of mRNA payload. A pre-calculated amount of HP was then injected into the PFHA-PEI-mRNA solution at 0.5 mL/s while PFHA-PEI-mRNA solution was being stirred at 500 RPM via the same RSM device to complete the formation of PFHA-PEI-mRNA-HP nanoparticles. Because injecting PFHA-PEI-mRNA directly into the HP solution would cause excessive binding of HP onto each individual PFHA-PEI-mRNA complex and result in overwhelming electrostatic binding competition between HP and mRNA, HP was injected into PFHA-PEI-mRNA solution at a slow speed to achieve the gradual HP surface embellishing on PFHA-PEI-mRNA (FIG. 1C).

### Physicochemical Property Characterization

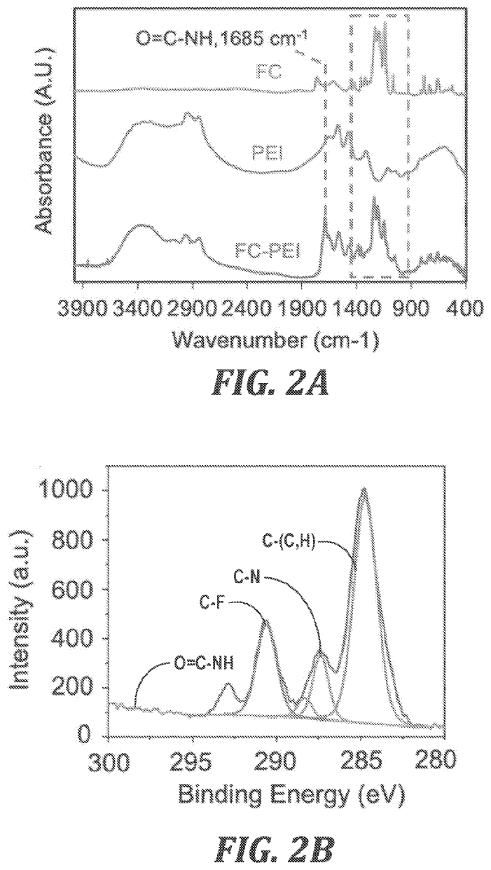

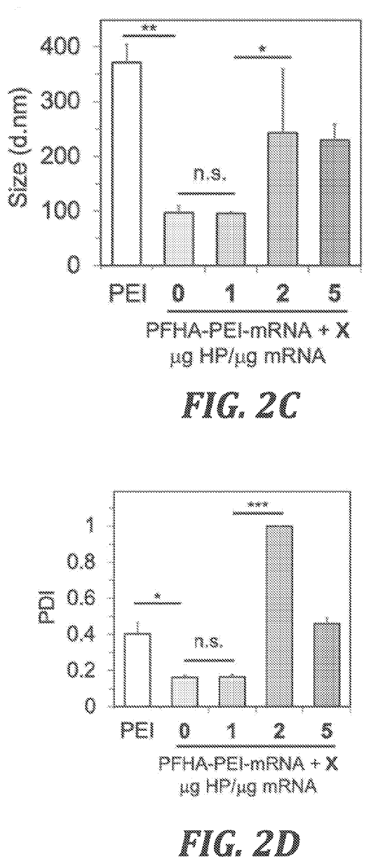

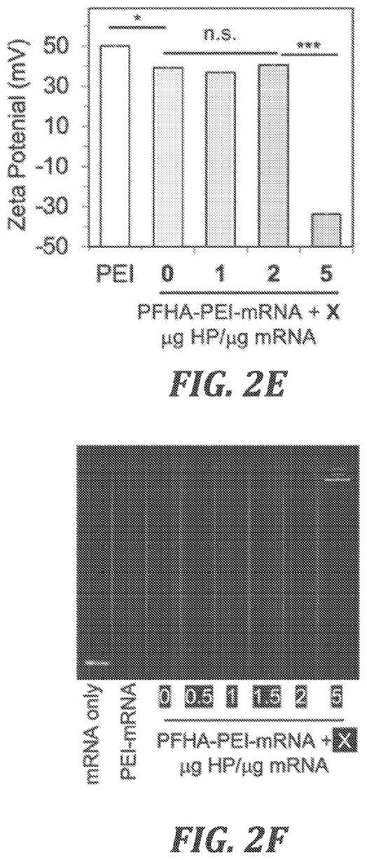

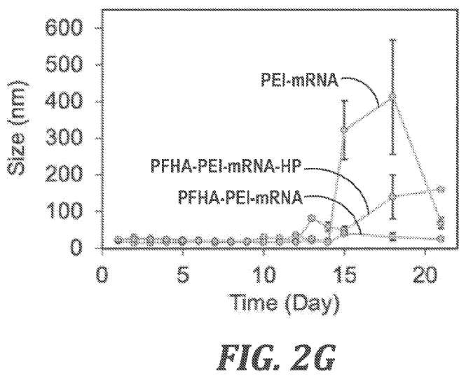

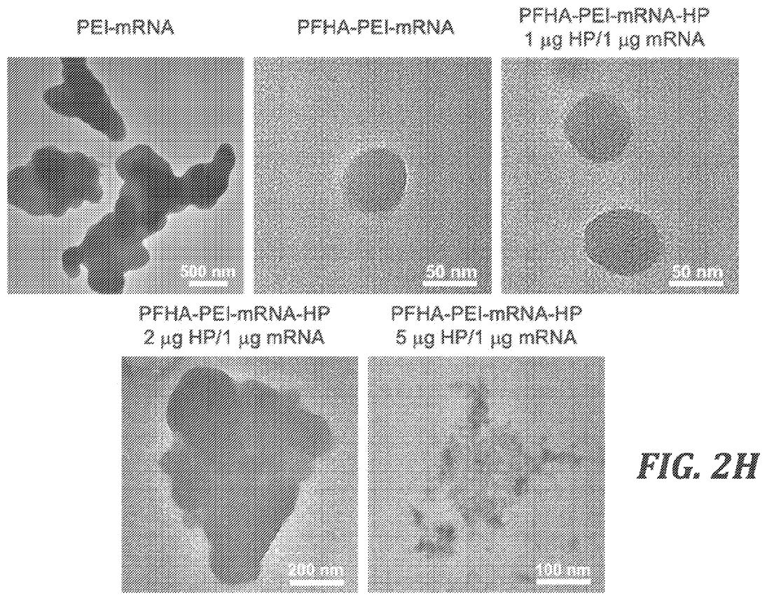

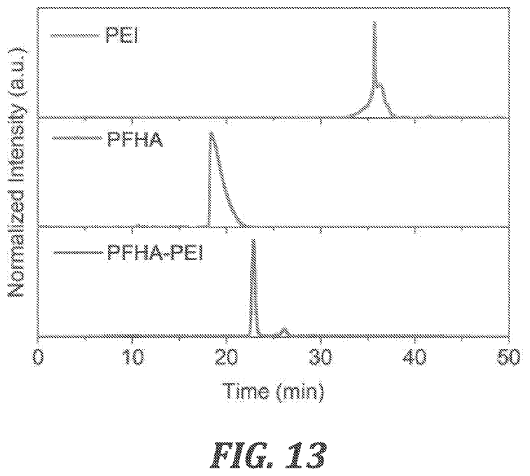

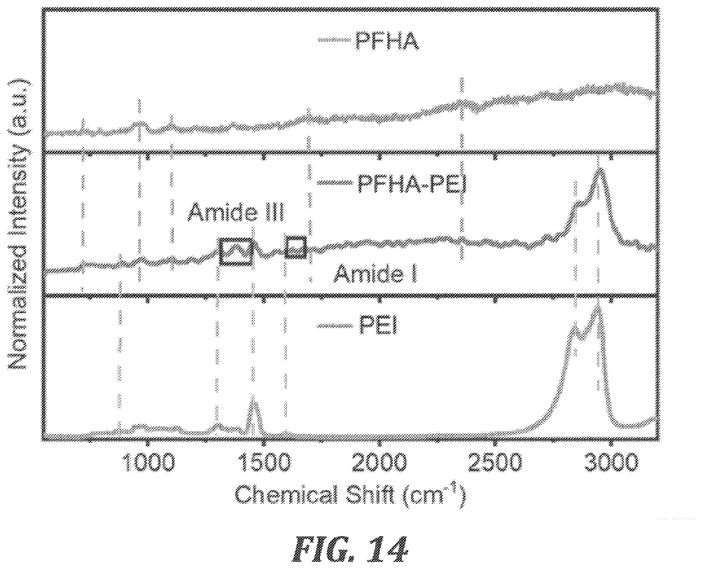

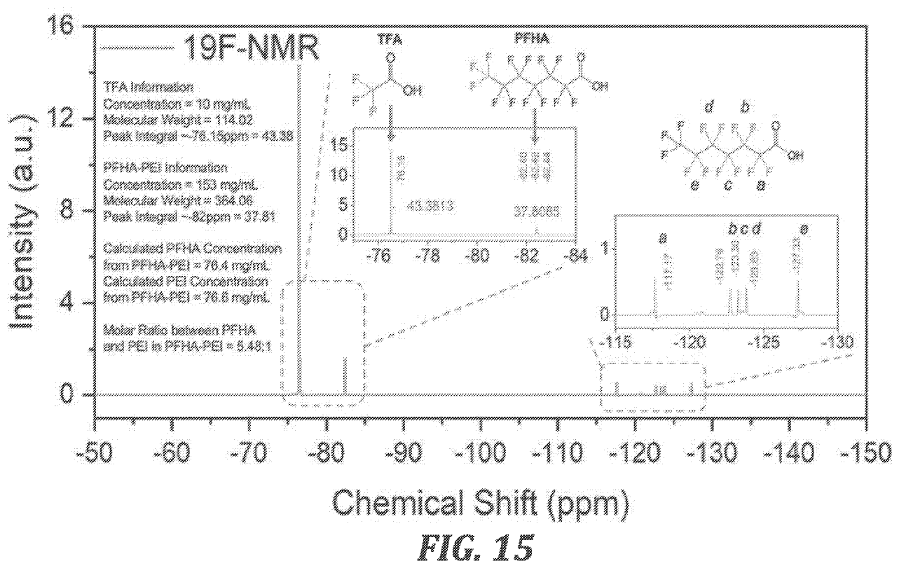

FTIR and XPS were performed on the purified PFHA-PEI product to confirm the presence of PFHA on PEI after conjugation. The purity of PFHA-PEI was evaluated by high performance liquid chromatography (HPLC). The retention time of PFHA, PFHA-PEI and PEI was 18, 23, and 36 minutes, respectively (FIG. 13). The fact that the PFHA-PEI spectrum did not contain noticeable peaks from pure PFHA and pure PEI suggests the high purity of PFHA-PEI. FTIR analysis revealed the amide bond formation between PFHA and PEI which was absent from the spectra of pure PFHA or PEI (FIG. 2A). The unique peak pattern of PFHA was also found adding to PEI's peak pattern in PFHA-PEI's spectrum, indicating successful conjugation of PFHA on PEI. XPS analysis (FIG. 2B) and Raman spectroscopy analysis (FIG. 14) of PFHA-PEI also confirmed the presence of the amide bond between PFHA and PEI. The fluorination degree of PEI was characterized by quantitative ¹⁹F NMR. With trifluoroacetic acid (TFA) with its characteristic —CF3 peak at −76.15 ppm serving as the internal standard, the unique —CF3 triplet peaks of PFHA on PFHA-PEI at around-82.4 ppm was used to calculate the fluorination degree of PEI. Quantitative results by comparing the integrated area under peaks of —CF3 from PFHA to that from TFA revealed that the PFHA:PEI molar ratio of PFHA-PEI is 5.48:1 (FIG. 15). These results collaboratively validated that the synthesis of PFHA-PEI was successful.

Size, surface charge, and shape all play roles in determining nanoparticle's cellular uptake amount, intracellular fate, and the eventual success of payload delivery. Spherical, cationic nanoparticles with 30-150 nm diameter have been shown to have balanced performance in blood/serum stability, cellular uptake amount and endosomal escape efficiency. Hence, hydrodynamic size and surface charge of PFHA-PEI-mRNA-HP nanoparticles were measured to study their suitability for intracellular mRNA delivery. The influence of each component of PFHA-PEI-mRNA-HP nanoparticle on its overall hydrodynamic size and surface charge were investigated. Without PFHA and HP, branched PEI with 2 kDa molecular weight alone could not effectively condense mRNA into a compact nanoparticle as PEI-mRNA is larger than 350 nm in diameter with high polydispersity index of >0.4 (FIGS. 2C and 2D). When PFHA is integrated into the system, PFHA-PEI was able to condense mRNA into a nanoparticle smaller than 100 nm in size with PDI<0.2. The further incorporation of HP (mRNA:HP wt/wt ratio of 1:1) did not increase the size and PDI of PFHA-PEI-mRNA nanoparticle, indicating that adding HP at this amount did not affect the compactness nor the uniformity of PFHA-PEI-mRNA nanoparticles. The zeta potential measurements yielded a value close to 40 mV for PFHA-PEI-mRNA and PFHA-PEI-mRNA-HP nanoparticles, and a value close to 50 mV for PEI-mRNA is close to 50 mV (FIG. 2E). It is reasonable that PEI-mRNA would possess slightly higher surface charge due to its much larger size than the other two nanoparticle formulations and hence would carry more positive charges. Although PFHA-PEI-mRNA is much smaller than PEI-mRNA, its zeta potential (between 35 and 40 mV) is only slightly lower than PEI-mRNA's. This phenomenon suggests that PFHA-PEI-mRNA possesses higher charge density than PEI-mRNA. Because structural compactness is challenging to maintain at high charge density due to the repulsion between same charges, additional favorable energy is required to overcome the structurally destabilizing electrostatic repulsion. The addition of a single component, PFHA, helps maintain the compactness of PEI-mRNA complex, indicating that PFHA's tendency to self-assemble could be the driving energy to overcome same charge repulsion in this system.

Because heparin is a polyanion that could compete with mRNA for electrostatic binding and induce the formation of large aggregates between cationic complex due to charge neutralization, the amount of HP in mRNA complexes was tuned. When added at the desired amount without affecting the overall stability of mRNA complexes, HP could partially shield positive charges on cationic mRNA complexes to increase biocompatibility and alleviate the binding tension between mRNA and cationic polymers to facilitate the release of mRNA for translation in cytoplasm. Nevertheless, over-adding HP can result in mRNA complex destabilization and possibly premature mRNA release. Therefore, different amounts of HP were added to PFHA-PEI-mRNA to create different versions of mRNA complexes to study the upper limit of HP at which PFHA-PEI-mRNA-HP complex would disintegrate. At or below 1:1 wt/wt of mRNA:HP, the results suggest that PFHA-PEI-mRNA-HP retained similar compact size and zeta potential to that of PFHA-PEI-mRNA (FIGS. 2C and 2D). The size started to increased slightly at 1:1.5 wt/wt of mRNA:HP, indicating slight destabilization in the compactness of PFHA-PEI-mRNA. At 1:2 wt/wt of mRNA:HP, PFHA-PEI-mRNA-HP's size drastically increased from sub-hundred nm to >240 nm (FIG. 2C). Even though the zeta potential of PFHA-PEI-mRNA-HP at 1:2 wt/wt of mRNA:HP remained at 40 mV, the much larger hydrodynamic size indicates that the charge density was significantly lower than that of nanoparticle at 1:1 wt/wt of mRNA:HP. These data could mean that HP started to destabilize PFHA-PEI-mRNA at mRNA 1:2 HP wt/wt and caused the formation of large aggregates. But mRNA remained largely unexposed as the zeta potential remained in highly positive realm. As the HP amount was further raised to mRNA 1:5 HP wt/wt, PFHA-PEI-mRNA-HP's zeta potential was completely reverted to the negative realm, suggesting the release of large anionic mRNA molecules and full disintegration of PFHA-PEI-mRNA-HP. The hydrodynamic and zeta potential results were corroborated by gel retardation assay. From the gel image (FIG. 2F), there were noticeable mRNA signals from the wells of PFHA-PEI-mRNA-HP at 1:5 wt/wt of mRNA:HP, which can be attributed to the partial exposure of the released mRNA from this sample. Meanwhile, there was no detectable signal in the wells loaded with PFHA-PEI-mRNA at other mRNA:HP ratios, suggesting that mRNA is well protected and unexposed in these samples. Transfection test with various HP amounts demonstrated that the optimal transfection results were obtained with 1:1 wt/wt of mRNA:HP wt/wt ratio on 3 different cell lines. Based on these results, PFHA-PEI-mRNA with 1:1 wt/wt of mRNA:HP can fully condense mRNA and is optimal in terms of size, zeta potential, and transfection efficiency.

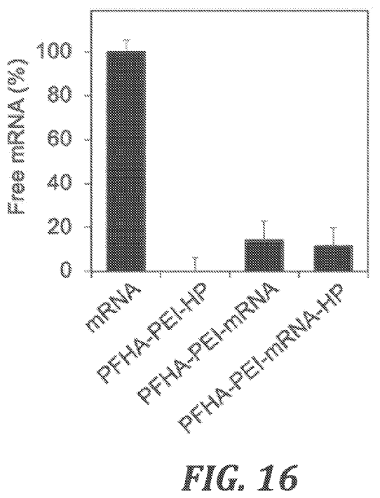

An mRNA encapsulation study was conducted with free mRNA as positive control and PFHA-PEI-HP (HP amount equivalent to that of mRNA:HP wt/wt ratio of 1:1) as mRNA free negative control. PFHA-PEI-mRNA-HP with mRNA:HP wt/wt ratio of 1:1 was selected as testing groups. The encapsulation results suggest that PFHA-PEI-mRNA-HP with mRNA:HP wt/wt ratio of 1:1 was able to achieve mRNA encapsulation efficiency of 89%, which is comparable to other concurrent highly efficient mRNA delivery vehicles (FIG. 16). Moreover, PFHA-PEI-mRNA-HP with mRNA:HP wt/wt ratio of 1:1 exhibited a serum stability greater than PEI-mRNA and PFHA-PEI-mRNA (FIG. 2G). PFHA-PEI-mRNA-HP was able to consistently retain its small size in serum-supplemented solution for over 21 days while PFHA-PEI-mRNA and PEI-mRNA showed unstable size fluctuation starting after day 13. This could be attributed to HP's contribution in shielding PFHA-PEI-mRNA-HP from excessive serum protein adsorption to prevent large aggregates. The fact that PEI-mRNA showed a much large size fluctuation than PFHA-PEI-mRNA suggests that PFHA also contributed to the serum stability of mRNA complex in this case.

TEM imaging was performed to provide visual confirmation of the shapes and sizes of PEI-mRNA, PFHA-PEI-mRNA and PFHA-PEI-mRNA-HP. From the hydrodynamic size measurement data, it was observed that the sequential addition of PFHA and HP at certain amount could help maintain desired compactness of mRNA-containing nanoparticles. Destabilization of these nanoparticles can occur at higher HP amount possibly due to the binding competition between the anionic HP and mRNA. The TEM imaging results were able to corroborate with these phenomena. Starting with just PEI-mRNA complex, the resultant structure was hundreds of nm in size with amorphous shapes (FIG. 2H). With PFHA incorporated, PFHA-PEI-mRNA was able to form a compact spherical nanostructure with diameter of around 50 nm. This significant change in structure could be attributed to PFHA's spontaneous self-assembly as described before. Adding HP at 1:1 wt/wt of mRNA:HP did not change the overall shape of PFHA-PEI-mRNA and only increased the diameter to slightly larger than 50 nm. TEM images with larger field of view confirmed that PFHA-PEI-mRNA and PFHA-PEI-mRNA-HP at 1:1 wt/wt of mRNA:HP were relatively monodispersed. Drastic structural changes of PFHA-PEI-mRNA-HP were observed when the mRNA:HP wt/wt ratio was further increased to 1:2 and eventually 1:5. At 1:2 wt/wt of mRNA:HP, aggregates with sizes far larger than 200 nm and irregular shape were observed (FIG. 2H). At 1:5 wt/wt of mRNA:HP, clear disintegration of PFHA-PEI-mRNA-HP was observed as there is not a distinct boundary of the nanostructure anymore (FIG. 2H). These imaging results agree with previous hydrodynamic size and zeta potential results as they all reveal the instability point of PFHA-PEI-mRNA-HP at 1:2 wt/wt of mRNA:HP and full disintegration at 1:5 wt/wt of mRNA:HP. Combined with the high mRNA loading efficiency and serum stability, PFHA-PEI-mRNA-HP with mRNA:HP wt/wt ratio of 1:1 was selected as the formulation for further study.

### Cellular Uptake and Endosomal Escape

For downstream transfection success, it is best to understand the intracellular trafficking mechanism of PFHA-PEI-mRNA-HP nanoparticle. As a fragile biomolecule prone to degrade, mRNA needs to be protected from RNases during transportation to cell surface, effectively ferried across cell plasma membrane, escape from endo-lysosome to avoid digestion, and eventually released into cytoplasm for translation. Although PFHA-PEI-mRNA-HP shows promising physicochemical properties, its intracellular fate is still largely unknown because nanoparticle's interaction with cells in biological medium is far too complex for mere size, shape, and surface charge profiles to dictate. The avoidance of trapping in digestive lysosomal compartments can be a hallmark of highly efficient transfection agent such as Lipofectamine. Therefore, understanding the intracellular trafficking pathway such as the mechanism of endosomal escape is essential for developing successful transfection agents.

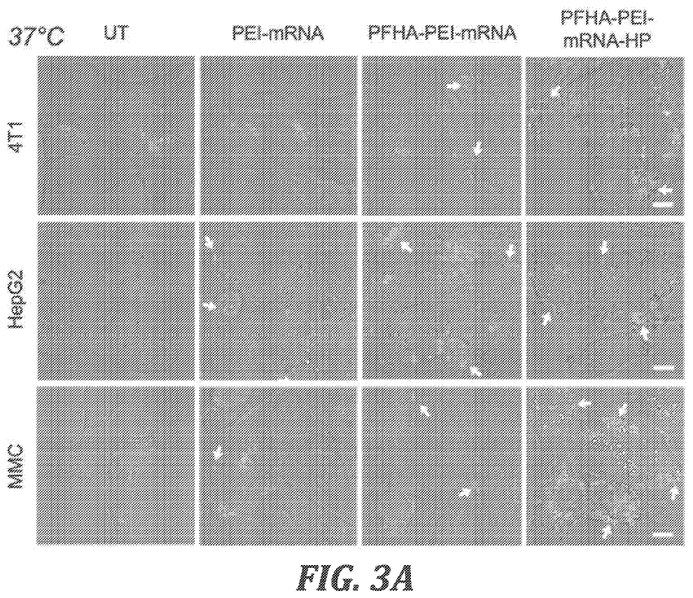

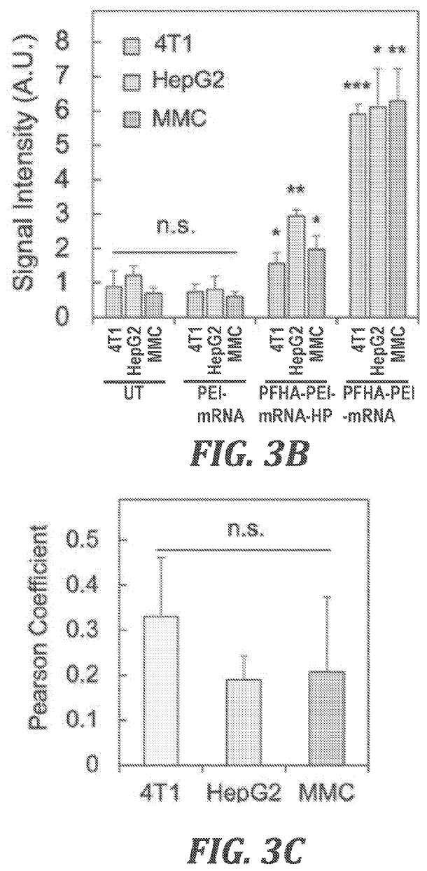

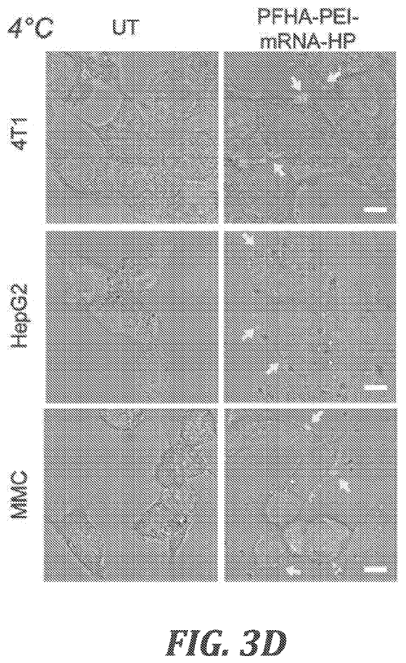

4T1 and MMC mouse breast cancer cells are chosen due to their capability to form syngeneic mouse tumors that closely mimic human breast tumors. In addition, HepG2 human liver cancer cell line is also chosen as it is extensively studied for oncogenesis and drug screening purposes. PEI-mRNA, PFHA-PEI-mRNA and PFHA-PEI-mRNA-HP with mRNA tagged with Cy5 fluorophores were incubated with 4T1, MMC and HepG2 cancer cells at 37° C. for 12 hours to understand how each component of PFHA-PEI-mRNA-HP can affect PFHA-PEI-mRNA-HP's intracellular fate. Lysotracker was added to cell culture 1 hour before the incubation period ends. From the imaging results, all the PEI-mRNA treated cells show negligible mRNA uptake at 12-hour time point (FIGS. 3A and 3B). It is possible that PEI-mRNA's size is too large to penetrate the plasma membrane. On the other hand, PFHA-PEI-mRNA demonstrates markedly higher cellular uptake in all 3 cell lines compared to PEI-mRNA. Because PFHA is prone to self-assemble and exhibits biphasic separation property in both aqueous and organic phase, the compact PFHA-PEI-mRNA can traverse lipid-water interface easily and in turn result in the pronounced cell uptake and endosomal escape. The further addition of HP significantly boosted the cell uptake while inheriting the quick endosomal escape characteristic of PFHA-PEI-mRNA. Heparin has been reported to be a cofactor or an independent ligand for fibroblast growth factor receptor (FGFR) whose expression is amplified in a variety of cancer cells, which could explain the prominent cell uptake enhancement effect caused by HP. Another possibility of this higher cell uptake is that HP slightly loosens PFHA-PEI's binding of mRNA so that mRNA is more exposed for fluorescent detection. The colocalization analysis further confirmed the endosomal escape of PFHA-PEI-mRNA. The Pearson colocalization coefficients between the Cy5-mRNA signal and the Lysotracker signal were 0.33, 0.19, and 0.21 in the PFHA-PEI-mRNA-treated 4T1, HepG2 and MMC cell images, respectively, suggesting that the majority of the mRNA from PFHA-PEI-mRNA-HP was able to escape from lysosome in all three cell lines (FIG. 3C).

A spherical nanoparticle with sub-hundred nanometer diameter and cationic surface charge typically enters cells via energy-dependent endocytosis. Because PFHA-PEI-mRNA-HP is a cationic spherical nanoparticle with sub-hundred nanometer diameter and simultaneously possesses hydrophobic moiety PFHA and cell receptor ligand HP, it is expected that PFHA-PEI-mRNA-HP would enter cells via the receptor-mediated energy-dependent endocytosis pathway. As energy-dependent pathways in cells are greatly inhibited at 4° C., the internalization of PFHA-PEI-mRNA-HP should be mostly halted at this temperature if endocytosis is responsible for cell uptake in this case. A cell uptake study where PFHA-PEI-mRNA-HP was applied to all three cell lines and incubate at 4° C. was conducted in parallel to the experiments conducted at 37° C. to validate this view. Compared to PFHA-PEI-mRNA-HP nanoparticles that were internalized into deep intracellular space when incubated with cells at 37° C., the imaging results from all 3 cell lines treated at 4° C. unanimously show that PFHA-PEI-mRNA-HP nanoparticles were either anchored on the surface of plasma membrane without internalization or only achieved shallow penetration into cytoplasm (FIG. 3D). Notably, the evident lysotracker signal presented in cells incubated at 37° C. mostly disappeared in cells incubated at 4° C. At 37° C., there was a weak lysotracker signal from the untreated group, showing the natural endocytosis process in the untreated cells. The lysotracker signal intensity increased significantly in the PFHA-PEI-mRNA-HP-treated cells. This phenomenon is expected as more endocytic vesicles are formed while the cells are actively endocytosing more exogenous materials (i.e., PFHA-PEI-mRNA-HP). The fact that the lysotracker signal was barely observable in both the untreated and the treated cells in the 4° C. panel could be the indicator of greatly suppressed endocytosis under this low temperature. These results collectively pointed out that even though PFHA-PEI-mRNA-HP could still bind to cell plasma membrane via either electrostatic adsorption or HP-FGFR interaction at lower temperature, it couldn't be efficiently internalized with endocytosis being effectively halted at 4° C. Therefore, it is clear that the energy-dependent endocytosis is primarily responsible for the cellular internalization of PFHA-PEI-mRNA-HP.

Another endosomal escape study was conducted with identical treatment conditions except that the incubation time was 2 hours instead of the previous 12 hours. This study aimed to investigate the early-stage cell uptake and endosomal escape and compare to the longer 12-hour time point. From the 2-hour time point results, the cellular uptake amount of PFHA-PEI-mRNA is significantly higher at 12h than at 2h, suggesting that PFHA-PEI-mRNA can be effectively deposited into intracellular space over time. The increase of PFHA-PEI-mRNA-HP's cellular uptake from 2h to 12h was even more pronounced compared to that of PFHA-PEI-mRNA, which further corroborates the facilitative utility of HP in cellular uptake. Notably, the distance separation between the mRNA signal and the lysotracker's signal from these two groups was clear even at the 2h time point, meaning that PFHA-PEI-mRNA and PFHA-PEI-mRNA-HP were efficient at cellular uptake and endo-lysosomal escape as they managed to effectively achieve both within 2 hours. PFHA-PEI-mRNA-HP's ability to continuously accumulate in cytoplasm over time and achieve quick endosomal escape in different types of cells could set the stage for successful mRNA transfection in various cell lines later.

### Biocompatibility Tests

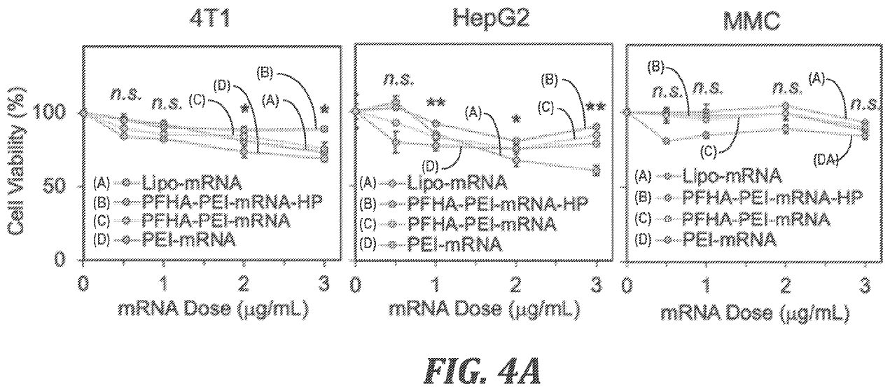

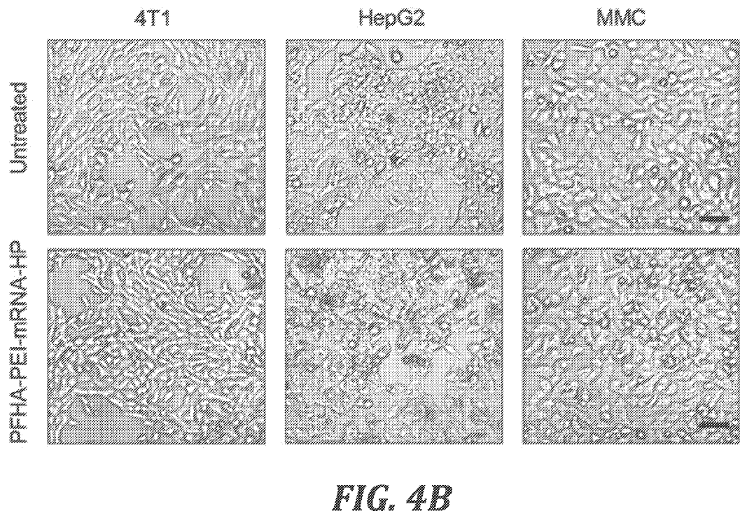

PEI-mRNA, PFHA-PEI-mRNA, PFHA-PEI-mRNA-HP and Lipofectamine 2000-mRNA were applied to 4T1, HepG2 and MMC cell lines to assess their biocompatibility based on the quantitative Alamar blue cell viability assay results and observations from bright field cell images. 4T1 cells treated with PFHA-PEI-mRNA-HP were able to retain around 90% viability across the mRNA concentration ranging from 0 to 3 mg/mL (FIG. 4A). On the other hand, Lipofectamine 2000-mRNA inflicted more than 20% viability loss on 4T1 at 2 mg/mL and above. The toxicity inflicted by PFHA-PEI-mRNA falls between those by PFHA-PEI-mRNA-HP and Lipofectamine 2000-mRNA while PEI-mRNA exerted the highest toxicity on 4T1 by reducing its viability to around 70% at 2 mg/mL and above. On HepG2 cell line, Lipofectamine 2000-mRNA exhibited a clear trend in its toxicity profile. As mRNA concentration increased from 0 to 3 mg/mL, HepG2 cells' viability decreased from 100% to around 70% and eventually 60% (FIG. 4A). PEI-mRNA treated HepG2 cells consistently showed around 75% viability at mRNA concentration between 0.5 to 3 mg/mL. On the other hand, PFHA-PEI-mRNA or PFHA-PEI-mRNA-HP-treated HepG2 cells were mostly able to retain >80% of viability across 0.5 to 3 mg/ml mRNA concentration. On MMC cell line, PFHA-PEI-mRNA, PFHA-PEI-mRNA-HP and Lipofectamine 2000-mRNA treatments all resulted in >95% cell viability when mRNA concentration is between 0 to 2 mg/mL (FIG. 4A). Cell viability slightly decreased to around 90% when mRNA concentration increased to 3 mg/mL. However, PEI-mRNA treated MMC cells' viability was consistently around 85%.

The quantitative cell viability assay results were corroborated by bright field images. The bright field images of the untreated or the PFHA-PEI-mRNA-HP-treated 4T1 cells showed similar cell density and morphology, which suggests that the 4T1's proliferation rate and health were not significantly affected by the presence of PFHA-PEI-mRNA-HP (FIG. 4B). 4T1 cells treated with PEI-mRNA, PFHA-PEI-mRNA and Lipofectamine 2000-mRNA showed slightly lower cell density than the untreated cells, agreeing with 4T1's cell viability results that these treatments had inflicted mild toxicity on 4T1 cells. Although the PFHA-PEI-mRNA-HP-treated HepG2 cells displayed similar cell density as the untreated cell, the morphology of the treated HepG2 appeared to be slightly clumpier and more corrugated than the untreated cells (FIG. 4B). This corresponds to the slight decrease of viability of the HepG2 cells treated by PFHA-PEI-mRNA-HP at 2 mg/mL mRNA. HepG2 cell images also confirmed that Lipofectamine 2000-mRNA indeed caused noticeable cytotoxicity to HepG2 cells as the cell density was significantly lower and cell morphology appeared to be clumpier. Meanwhile, PEI-mRNA and PFHA-PEI-mRNA only displayed mild adverse effects on HepG2 cells as the cell viability results suggest. Bright field images of MMC cells did not show noticeable differences in terms of cell density and morphology between the PFHA-PEI-mRNA-HP-treated and the untreated cells, which agrees well with the cell viability test results (FIG. 4B). PEI-mRNA, PFHA-PEI-mRNA and Lipofectamine 2000-mRNA did not show observable impacts on MMC's cell density and morphology.

Additionally, PFHA-PEI-mRNA-HP's biocompatibility was also evaluated in vivo. The PFHA-PEI-mRNA-HP-treated mice and the untreated mice showed similar levels of glucose, creatinine, blood urea nitrogen, salts, proteins and aspartate transaminase. Moreover, The PFHA-PEI-mRNA-HP-treated mice and the untreated mice were also stable in maintaining healthy body weight for two weeks post injection. These results suggest that PFHA-PEI-mRNA-HP did not inflict observable tissue toxicity in vivo.

Taking the biocompatibility data together, PFHA-PEI-mRNA-HP and PFHA-PEI-mRNA both displayed promising biocompatibility on all 3 cell lines because they typically inflict less than 20% growth retardation even at mRNA concentration as high as 3 mg/mL. The fact that PFHA-PEI-mRNA-HP-treated cells consistently showed slightly higher viability than that treated by PFHA-PEI-mRNA could suggest HP's contribution in improving mRNA complex's biocompatibility. Without PFHA and HP, PEI-mRNA's toxicity could be obvious on some cell lines. These results indicate that HP and PFHA are both beneficial in alleviating the toxicity from PEI. Although Lipofectamine 2000-mRNA showed decent biocompatibility on 4T1 and MMC cells, it inflicted noticeable toxicity on HepG2 cells at elevated mRNA concentrations so that it can pose safety concerns when applied to certain cell types. PFHA-PEI-mRNA-HP also showed promising results in biocompatibility test in mice, suggesting that PFHA-PEI-mRNA-HP could be safe for future in vivo applications.

### Transfection Efficiency

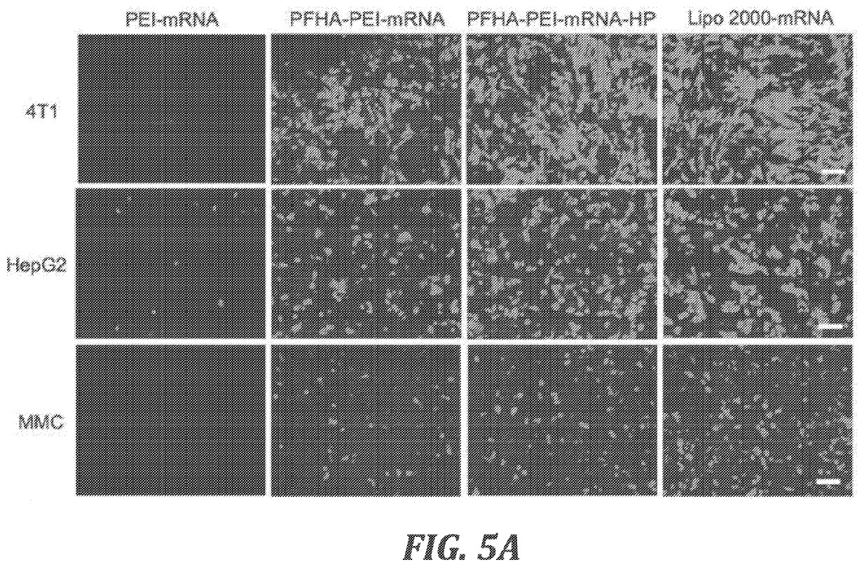

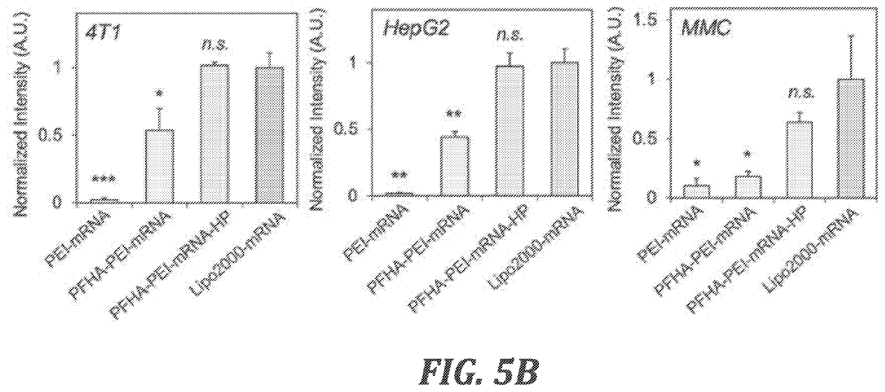

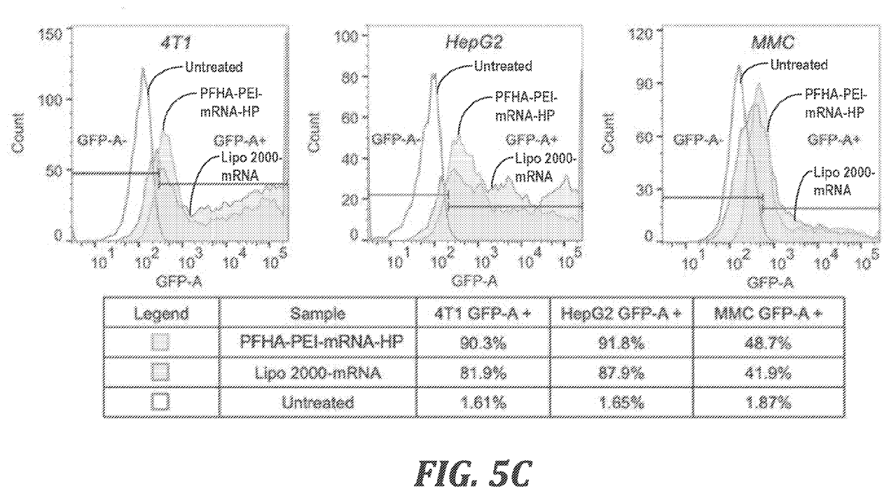

PEI-mRNA, PFHA-PEI-mRNA, PFHA-PEI-mRNA-HP nanoparticles were applied to 4T1, HepG2, and MMC cell lines to test how each component of PFHA-PEI-mRNA-HP could affect transfection outcomes. The mRNA dosages needed to achieve optimal transfection efficiency on different cell types were determined by dose sensitivity study. Through the dose sensitivity study, the mRNA concentration for transfecting all cancer cells was set at 2 mg/mL. As a “gold standard” of commercially available transfection agent touting high transfection efficiency and safety, Lipofectamine 2000-mRNA lipoplex was used as a positive control for comparison. Based on the fluorescent image results (FIGS. 5A and 5B), the incorporation of PFHA into PEI-mRNA significantly boosted the mRNA transfection efficiency in all cell lines. The conspicuous improvement in transfection could be attributed to PFHA's inertness, hydrophobicity as well as its lipophobicity. Because it is unfavorable for PFHA to interact with neither polar nor non-polar environment, PFHA can self-assemble into compact structures with itself and remain inert to its ambience. These characteristics render PFHA suitable for protecting fragile payloads such as easily degraded mRNA. The addition of HP to PFHA-PEI-mRNA further significantly enhanced the transfection efficiency on all cell lines. As observed in the previous intracellular trafficking results, the presence of HP significantly increases the cellular uptake of PFHA-PEI-mRNA-HP compared to its counterpart without HP, corroborating HP's enhancement effect on transfection. The mechanism behind HP boosting transfection could be due to the synergy between the FGFR-HP interaction and the slight loosening of mRNA binding to a subtle packing-unpacking balance to promote mRNA release for translation in cytoplasm while retaining sufficient mRNA protection during transportation. The transfection images show that PFHA-PEI-mRNA-HP was able to achieve comparable transfection efficiency to that of Lipofectamine 2000-mRNA on 4T1, HepG2 and MMC cell lines. These image data combined with the physicochemical profiles of PFHA-PEI-mRNA-HP collectively showcase the usefulness of well-rounded attributes in size, shape, surface charge, biocompatibility, and intracellular trafficking profiles in successful mRNA transfection. Quantitative flow cytometric analysis was performed to study the percentage of successful transfection cell population from each cancer cell line (FIG. 5C). Similarly, PFHA-PEI-mRNA-HP's transfection performance was compared to Lipofectamine 2000-mRNA in this study. The flow cytometry results showed that PFHA-PEI-mRNA-HP was able to achieve 90.3% and 91.8% transfection efficiency compared to Lipofectamine 2000-mRNA's slightly lower 81.9% and 87.9% on 4T1 and HepG2 cell lines, respectively. Meanwhile, PFHA-PEI-mRNA-HP was also able to transfect 48.7% of MMC cell population, compared to Lipofectamine 2000-mRNA's 41.9%.

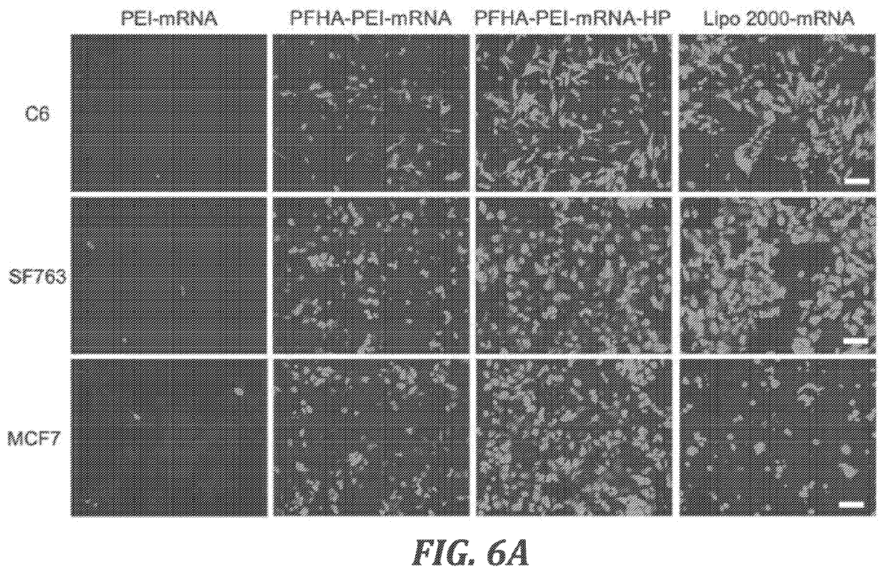

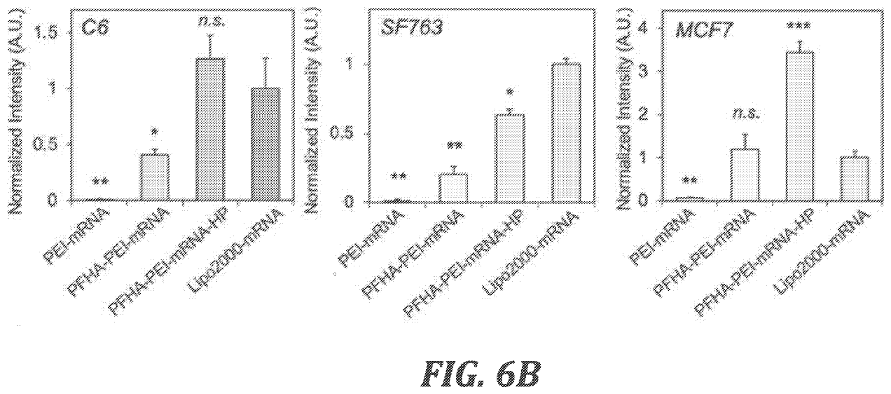

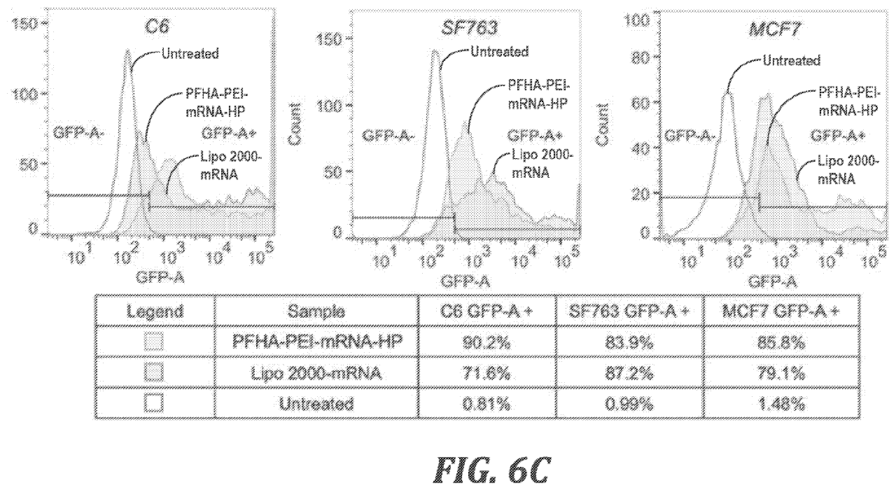

Human breast cancer MCF7 cell line, human brain cancer SF763 cell line, and rat brain cancer C6 cell lines have also been extensively applied in cancer research. These cell lines were also subjected to transfection to further validate the wide applicability of PFHA-PEI-mRNA-HP on various types of cancer cells. Similar to the transfection results observed from 4T1, HepG2, and MMC cells, the addition of PFHA and HP to PEI-mRNA significantly enhanced the transfection efficiency on MCF7, SF763, and C6 cells (FIGS. 6A and 6B). The flow cytometry analysis showed that PFHA-PEI-mRNA-HP reliably achieved high transfection efficiency of 90.3%, 83.9%, and 85.8%, compared to Lipofectamine 2000-mRNA's 71.6%, 87.2%, and 79.1%, on MCF7, SF763, and C6 cells respectively (FIG. 6C).

Based on the flow cytometry results, PFHA-PEI-mRNA-HP slightly outperformed Lipofectamine 2000-mRNA's transfection on 5 cell lines except for SF763 and exhibited high transfection efficiency (>80%) on 5 cell lines except for MMC. Lipofectamine 2000-mRNA's transfection efficiency on MMC was also significantly suppressed. The reason behind MMC's lower transfection efficiency remains uncertain even though cell uptake and endosomal escape of PFHA-PEI-mRNA-HP was highly efficient in MMC cells.

### Functionality Test After Above 0° C. Storage

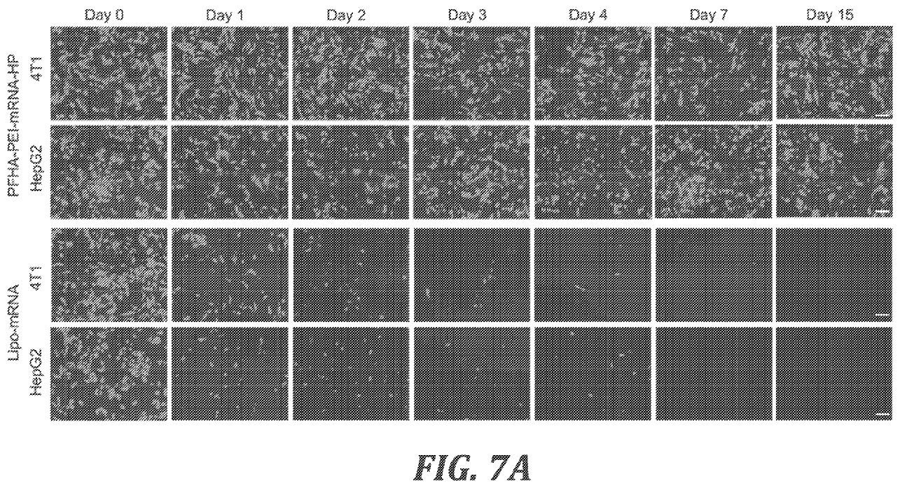

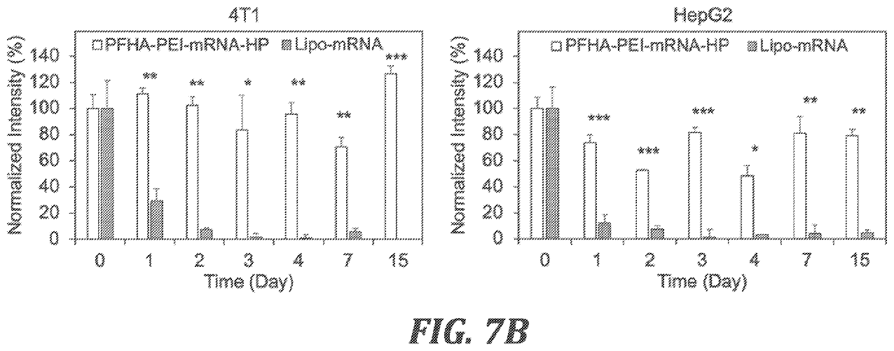

mRNA's labile nature brings extra challenges to the storage of mRNA products as mRNA is highly susceptible to nucleases, oxidation, and hydrolysis. Common storage condition for mRNA complexes, such as the COVID-19 mRNA vaccines developed by Pfizer-BioNTech and Moderna, usually require deep-freezing at −80° C. or −20° C. These vaccines not only are costly to distribute in cold-chain transportation but also only have narrow window to be administered once thawed, which is usually within hours because frequent freeze-thaw cycle could easily jeopardize the structural integrity of mRNA. It would bring great convenience to the usage and transportation of mRNA complexes if they can be stably stored unfrozen at above 0° C. Even though lyophilization has been reported to significantly improve mRNA complex's stability at above 0° C., the additional cost and labor for lyophilization and reconstitution later plus the quality control between these steps may bring more challenges and uncertainties for large-scale processing. To test if storing PFHA-PEI-mRNA-HP solution at above 0° C. would jeopardize mRNA stability and transfection functionality, PFHA-PEI-mRNA-HP was either refrigerated at 4° C. PFHA-PEI-mRNA-HP samples stored at 4° C. were then applied to 4T1 and HepG2 cells for transfection on day 0 (the same day the samples were prepared) as well as on day 1, day 2, day 3, day 4, day 7, and day 15 post sample preparation. Lipofectamine 2000-mRNA was also prepared and stored and applied similarly for comparison. The refrigerated PFHA-PEI-mRNA-HP and Lipofectamine 2000-mRNA sample were allowed to be equilibrated to room temperature before applied to cell culture each time. The results showed that PFHA-PEI-mRNA-HP stored at 4° C. did not show any significant compromise in transfection efficiency on both 4T1 and HepG2 cells for 15 days, indicating that mRNA was well-protected by the PFHA-PEI-HP construct and was able to maintain its structural stability and functionality for prolonged period of time at 4° C. (FIG. 7A). On the other hand, the Lipofectamine 2000-mRNA showed significant decrease in transfection efficiency on both cell lines after just one day being stored at 4° C. Lipofectamine 2000-mRNA lost most of its transfection efficiency after two days of refrigeration, suggesting the Lipofectamine 2000-mRNA is unstable while being stored at 4° C. Quantitatively, PFHA-PEI-mRNA-HP showed negligible loss of its transfection efficiency on 4T1 cells for 15 days, whereas Lipo-mRNA lost more than 70% of its transfection efficiency on day 1 and further lost 20% more so that the transfection efficiency was only around 5% of that on day 0 between day 2 and 7 (FIG. 7B). On HepG2 cells, PFHA-PEI-mRNA-HP was able to retain 80% of its transfection efficiency even at day 15 even though the transfection efficiency fluctuated during the study which could be due to variation of HepG2 conditions. On the other hand, Lipo-mRNA nearly lost 90% of its transfection efficiency on HepG2 cells on day 1 and was never able to recover. Besides hydrolysis, a recent study also showed that the adduct formation between ionizable lipids and mRNA at temperature above 0° C. could compromise the structural integrity of mRNA and cause suppressed protein expression. Because ionizable lipids are indispensable components in virtually all LNPs, the ionizable lipid-mRNA adduct formation could be one of the factors causing the quick decline in functionality of Lipofectamine 2000-mRNA stored above 0° C.

### In Vivo Transfection Results in a Triple Negative Breast Tumor Model

The effectiveness of mRNA delivery for the polymeric mRNA delivery platform described herein (i.e., PFHA-PEI-mRNA-HP) was demonstrated to suppress triple negative breast cancer tumor growth in vivo.

Targeting Ligand Equipped PFHA-PEI-LHP for the Co-Delivery of Therapeutic mRNAs (IL12 Interleukin 12 mRNA and IFNγ-Interferon Gamma mRNA to Suppress TNBCs In Vivo.

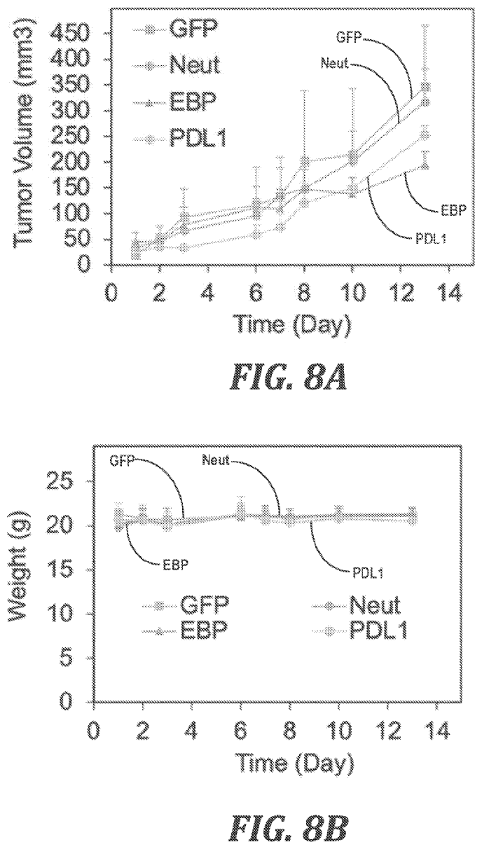

Triple negative breast tumor model was established by inoculating M6 murine triple negative breast cancer cells subcutaneously into C3 (1)-tag mice. The M6 tumor model was used to evaluate the in vivo therapeutic effects of the tumor target ligand (EBP, endoglin-binding peptide) and anti-PDL1-functionalized PFHA-PEI-IL12+IFNγ mRNA-HP with PFHA-PEI-GFP mRNA-HP-Neut (GFP) as material control and PFHA-PEI-IL12+IFNγ mRNA-LHP-Neut (Neut) as ligand free control. 15 mg mRNA/mouse of these four types of mRNA complexes were injected into M6 tumor-bearing mice after the tumor reached 50 mm³ three times every two days in between injections. Tumor volumes and body weights of the treated mice were monitored throughout the study. From the therapeutic results, both EBP and anti-PDL1 conjugated mRNA NP showed significant tumor growth suppression compared to material control and targeting ligand free control groups. Specifically, EBP conjugated mRNA NPs showed the best results by suppressing more than 40% of TNBC tumors at the end of study followed by near 30% by anti-PDL1 conjugated mRNA nanoparticles compared to the material control group (FIG. 8A). The tumor growth from the ligand free control is similar to that of the material control group, suggesting that targeting ligand plays a role in the therapeutic effect of mRNA nanoparticles. There were no significant body weight fluctuations from all the groups observed during the study, indicating that there is no acute toxicity resulted from the treatment (FIG. 8B).

### Stem Cell Transfection

In addition to transfection of cancer cells, the polymeric mRNA delivery nanoplatform described herein is effective for transfecting human induced pluripotent stem cells (hiPSCs).

The current approach of directing hiPSCs differentiation pathways often involves viral gene transduction which poses certain risks such as cytotoxicity, insertional mutagenesis and immunogenicity. To render the application of hiPSC safer in regenerative medicine, non-viral gene delivery vehicles are needed to direct hiPSCs' differentiation instead. Therefore, PFHA-PEI-mRNA-LHP was also applied to hiPSC. Notably, PFHA-PEI-mRNA-LHP was able to achieve ultra-high mRNA transfection efficiency (>90%) on certain types of breast cancer cells, brain cancer cells and liver cancer cells while also able to show significantly higher transfection than Lipofectamine 2000 on human induced pluripotent cells (hiPSCs).

### Cellular Uptake and Endosomal Escape.

PEI-mRNA, PFHA-PEI-mRNA and PFHA-PEI-mRNA-LHP with mRNA tagged with Cy5 fluorophores were incubated with hiPSCs at 37° C. for either 3 hours or 13 hours to understand how each component of PFHA-PEI-mRNA-LHP can affect PFHA-PEI-mRNA-LHP's intracellular fate.

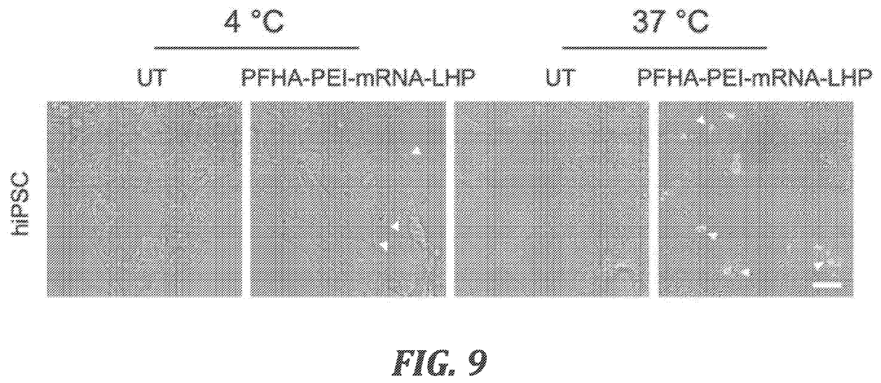

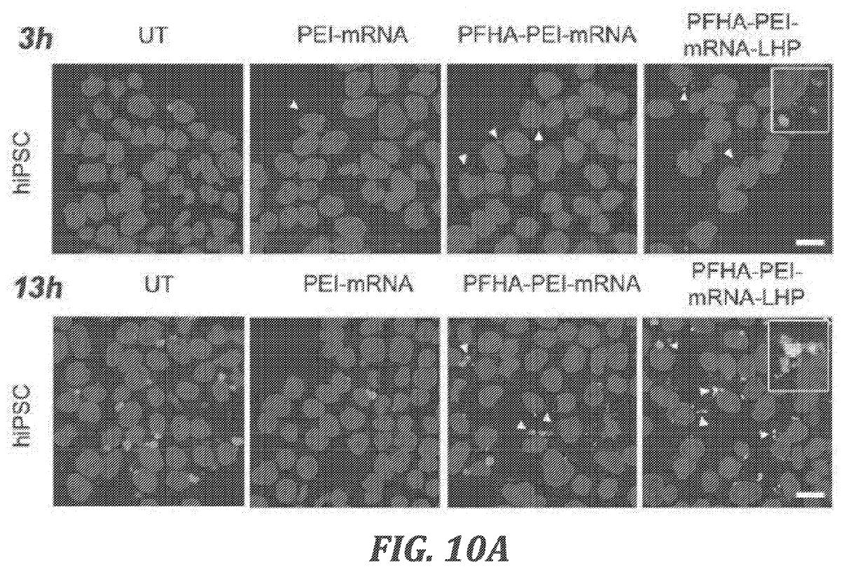

FIG. 9 compares cellular uptake and endosomal escape studies on human induced pluripotent stem cells (hiSPCs) at 4° C. or 37° C. Cy5-labeled mRNA was complexed with PFHA-PEI-LHP and added to cell cultures at 2 mg/mL mRNA dosage and incubated for 12 hours at either 4° C. or 37° C. before adding Lysotracker Red reagent and incubated for another 1 hours at their corresponding temperatures. mRNA-Cy5 signals, Lysotracker, and DAPI nuclear stain are shown with bright field images shown in background. Scale bar is 20 mm. In the 4° C. PFHA-PEI-mRNA-LHP images, arrows point at the locations where PFHA-PEI-mRNA-LHP nanoparticles are stranded on cell plasma membrane. In the 37° C. PFHA-PEI-mRNA-LHP images, arrows point at PFHA-PEI-mRNA-LHP nanoparticles which are deeply internalized and separated from endo-lysosomes.

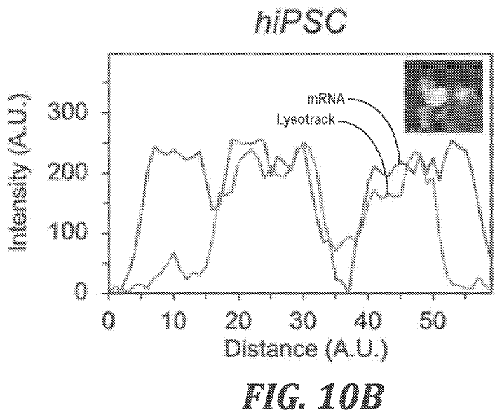

FIGS. 10A and 10B compare endosomal escape studies on hiPSCs at 37° C. at 12-h time points (3 h and 13 h). Confocal images of hiPSCs treated with PEI, PFHA-PEI, and PFHA-PEI-LHP are shown in FIG. 10A. Cy5-labeled mRNA was complexed with and added to cell cultures at 2 mg/mL and incubated at 37° C. for 12 hours or before adding Lysotracker Red reagent and incubated for another 1 hour. mRNA-Cy5 signal, Lysotracker, and DAPI nuclear stain are shown. Scale bar is 20 mm. Arrows point at the regions where distinguished separation between mRNA signal and Lysotracker signal. One representative region in the PFHA-PEI-mRNA-LHP image from each cell line is magnified into a separate image (the most right column). The dashed lines in the magnified images correspond to the distance where fluorescence intensity line profiles were analyzed. Signal intensity line profile analysis over the distance indicated by the dashed lines in the magnified images from the PFHA-PEI-mRNA-LHP panel are shown in FIG. 10B.

TRANSFECTION efficiency.

In addition to cancer cells, hiPSCs were also transfected to see if PFHA-PEI-mRNA-LHP has the potential to deliver mRNA to stem cells. The mRNA dosage for hiPSC transection was set at 1 μg/mL based on the dose sensitivity study. Similar to the transfection results of cancer cells, PEI-mRNA only showed negligible transfection on hiSPCs. Adding PFHA and then LHP to the mRNA complex drastically improve transfection performance on hiPSC. In this case, PFHA-PEI-mRNA-LHP displayed significantly higher transfection efficiency than Lipofectamine 2000-mRNA on hiPSC, confirming PFHA-PEI-mRNA-LHP's promising utility in transfecting stem cells. The fact that PFHA-PEI-mRNA-LHP could achieve higher transfection efficiency than the commercial agent Lipofectamine 2000 not only on cancer cells but also on stem cells implicates that PFHA-PEI-mRNA-LHP could be a reliable mRNA delivery platform for both cancer treatment and regenerative medicine involving stem cells.

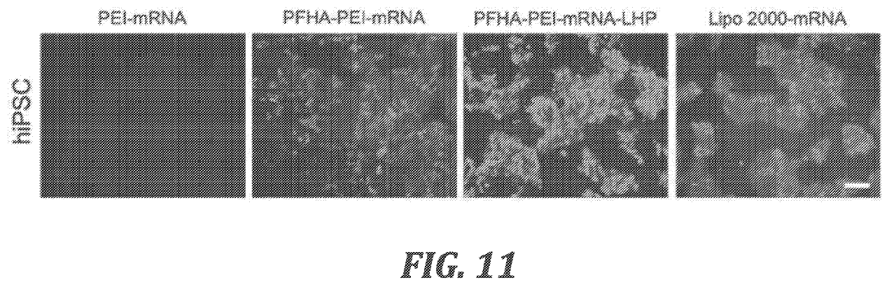

FIG. 11 compares transfection results on hiPSCs and illustrates transfection images of PEI-mRNA, PFHA-PEI-mRNA, and PFHA-PEI-mRNA-LHP with Lipofectamine 2000-mRNA as positive control on hiPSCs. Scale bar is 100 mm.

### Biocompatibility.

Lipofectamine 2000-mRNA inflicted significant toxicity as hiPSC's viability first dropped to around 70% at 0.5 and 1 μg/mL mRNA and plummeted to only around 30% when mRNA dose further increased to above 2 μg/mL. PEI-mRNA also showed explicit toxicity on hiPSC as PEI-mRNA-treated hiPSCs' viability first decreased to 60% and then to 40% as mRNA concentration increases. Meanwhile, PFHA-PEI-mRNA-treated hiPSC exhibited viability around 80% between 0.5 and 3 μg/mL mRNA doses. PFHA-PEI-mRNA-LHP was able to achieve >90% viability on hiPSC at 0.5 ug/mL mRNA and a consistent >80% viability between 1 and 3 μg/mL mRNA doses.

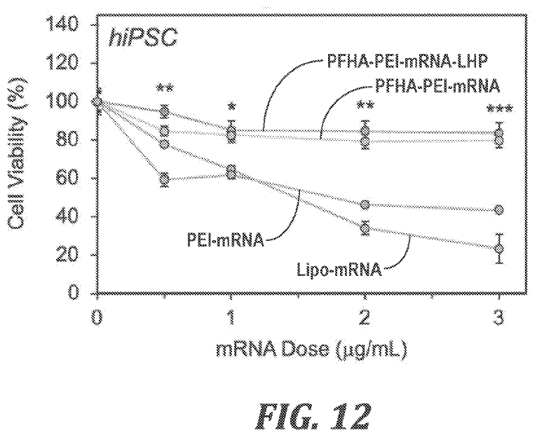

FIG. 12 compares cell viability test results on hiPSCs. PEI-mRNA, PFHA-PEI-mRNA, PFHA-PEI-mRNA-LHP and Lipofectamine 2000-mRNA were applied to hiPSCs at 0.5, 1, 2 and 3 mg/mL for 24 hours before Alamer blue assay was performed. The untreated cells' viability was normalized to 100%. Statistical analysis was performed to determine if the difference between the data points from the Lipo-mRNA-treated cells and the data points from the PFHA-PEI-mRNA-LHP treated cells were significant.

### INDUSTRIAL APPLICABILITY

Although lipid nanoparticle (LNP) platforms are presently the most advanced clinical mRNA delivery platform and have been extensively applied in vaccine formulations, the application of LNP-delivered mRNA (LNP-mRNA) is not without problems. The adverse effects caused by LNP-mRNA in clinic settings have raised public concern about its safety. The complex assembling process and stringent storage requirements also brought tremendous challenges in the manufacture and distribution of LNP-mRNA. Furthermore, the functionalization of LNP is limited due to scarce reactive sites on lipids. As an alternative to LNP, the present disclosure provides a polymeric mRNA delivery platform (“PFHA-PEI-mRNA-HP”) by simultaneously fluorinating and heparinizing low molecular weight PEI which can then be readily assembled with mRNA via a simple rotor-syringe mixing device. PFHA-PEI-mRNA-HP is a spherical cationic nanoparticle with sub-hundred nm diameter. From in vitro testing results with multiple cancer cell lines and stem cells, PFHA-PEI-mRNA-HP exhibited improved biocompatibility, transfection efficiency and storage stability than the commercial lipidic transfection agent Lipofectamine. The polymeric mRNA delivery platform described herein demonstrates the promising utility of functionalized cationic polymers in the field of mRNA delivery.

As used herein the term “about” refers to ±5% of the specified value.

### Materials and Methods

### Materials

CleanCap® EGFP mRNA was purchased from TriLink Biotechnologies (San Diego, CA, USA). Low molecular weight heparin was purchased from Galen Laboratory Supplies (North Haven, CT, USA). Branched PEI (MW 2 kDa) was purchased from Polysciences (Warrington, PA. USA). Microliter syringes (100 μL max volume) and removable needles (32 gauge, point style 3) were purchased from Hamilton (Reno, NV, USA). NE-300 “Just Infusion” TM Syringe Pump was purchased from New Era Pump System Inc. (Farmingdale, NY, USA). (1-Ethyl-3-[3-dimethylaminopropyl] carbodiimide hydrochloride (EDC), N-hydroxysuccinimide (NHS), Lab-Tek™ II 8-well chambered coverglass, NucBlue DAPI reagent, Lipofectamine 2000, LysoTracker™ Red DND-99, ultrapure agarose, antibiotic-antimycotic (100×), Tryple Express Enzyme solution, RPMI 1640 and DMEM cell culture medium were purchased from Invitrogen (Carlsbad, CA, USA). HyClone characterized fetal bovine serum (FBS) were purchased from GE Healthcare Life Sciences (Pittsburgh, PA, USA). Label IT Tracker Intracellular Nucleic Acid Labeling Kits were purchased from Mirus Bio (Madison, WI, USA). Single Strand RNA ladder was purchased from New England Biolabs (Ipswich, MA, USA). SpectraPOR7 dialysis membrane was purchased from Repligen Corp (Waltham, MA, USA). All other chemicals were purchased from Sigma-Aldrich (St Louis, MO, USA). 4T1, HepG2, MCF7, SF763, and C6 cell lines were purchased from American Type Culture Collection (Manassas, VA, USA). MMC cell line was kindly shared by the Disis group from Cancer Vaccine Institute at UW Medicine.

### Synthesis of PFHA-PEI

PFHA was conjugated onto PEI via EDC/NHS coupling chemistry. 127.4 mg of PFHA, 80.5 mg of EDC, and 58.1 mg of NHS were separately dissolved in methanol at 50 mg/mL concentration. PFHA, EDC, and NHS solutions were mixed together by adding EDC and subsequently NHS to PFHA solution. The mixed solution was placed on a rocker and incubated for 3 hours at room temperature. Next. 100 mg branched PEI was dissolved in methanol at 50 mg/mL and added to the PFHA-EDC-NHS mixture solution and rocked at room temperature for 16 hours. The resultant solution was dialyzed against Milli-Q water for 2 days using 1k MWCO SpectraPOR7 dialysis membrane. The dialyzed solution was centrifuged at 4000G for 5 mins to precipitate out large aggregates. The clear supernatant was then freeze-dried and stored at −20° C. for long term storage. The typical yield of a PFHA-PEI batch is around 60% of the combined mass of all the reactants.

### FTIR Spectra Collection

2 mg of each of the PFHA, PEI, and PFHA-PEI dry samples were mixed with 200 mg of KBr and pulverized into fine powders, and a pellet was prepared for characterization. FTIR spectra were obtained using a Nicolet 5-DXB FTIR spectrometer (ThermoFisher, Boston, MA) with a resolution of 4 cm-1 and averaging 64 runs.

### XPS Spectra Analysis

X-ray photoelectron spectroscopy (XPS, AXIS Ultra DLD/Surface Science Instruments S-Probe, Kratos) was performed to study the amide group (O═C—NH—) formation. This instrument has a monochromatized Al Ka X-ray and a low-energy electron flood gun for charge neutralization. The X-ray spot size for these acquisitions was on the order of 700×300 μm. The electrostatic lens was used for data collection. The pressure in the analytical chamber during spectral acquisition was less than 5×10⁻⁹ Torr. The pass energy for survey spectra (composition) was 160 eV. The pass energy for the high-resolution spectra was 40 eV. The take-off angle (the angle between the sample normal and the input axis of the energy analyzer) was 0° (0-degree take-off angle ˜100 Å sampling depth). The Kratos Vision2 software was used to determine the peak areas and to calculate the elemental compositions from the peak areas. CasaXPS was used to peak fit the high-resolution spectra. For the high-resolution spectra, a Shirley background was used, and all binding energies were referenced to the C Is C—C bonds at 285.0 eV.

### Formation of PFHA-PEI-mRNA-HP Complex

PFHA-PEI was redissolved in Milli-Q water at 10 mg/mL and was centrifuged at 16,000 G for 10 mins to eliminate possible large aggregates. The supernatant from PFHA-PEI was diluted to 7.5 mg/mL by 20 mM Hepes buffer (pH 7.4). mRNA was diluted to 0.5 mg/mL in 20 mM Hepes buffer (pH 7.4). HP was dissolved in 20 mM Hepes buffer (pH 7.4) at 0.5 mg/mL concentration. To make a PFHA-PEI-mRNA complex. 5 μL of mRNA solution was mixed with 5 μL of PFHA-PEI solution via the RSM device. Specifically, 5 μL of PFHA-PEI solution was first added to the bottom of a 0.6 mL microtube and 5 μL of mRNA solution was loaded into a Hamilton microliter syringe. mRNA solution was then slowly injected into PFHA-PEI solution at the flow rate of 1 mL/s controlled by a syringe pump while the PFHA-PEI solution was being stirred by a rotor tip at 500 RPM to ensure homogenous mixing. To add HP to PFHA-PEI-mRNA complex, desired amount of HP was loaded into a Hamilton microliter syringe and slowly injected into PFHA-PEI-mRNA solution at the flow rate of 0.5 mL/s controlled by a syringe pump while the PFHA-PEI-mRNA solution was being stirred by a rotor tip at 500 RPM to ensure homogenous mixing. For making the PEI-mRNA complex, PEI was first dissolved in 20 mM Hepes buffer (pH 7.4) at 7.5 mg/mL concentration followed by the same mixing procedure as that of making PFHA-PEI-mRNA complex.

### Hydrodynamic Size, Serum Stability, and Zeta Potential Measurement

The hydrodynamic size and zeta potential of PEI-mRNA, PFHA-PEI-mRNA and PFHA-PEI-mRNA-HP (with varying HP amounts) were determined using a Zetasizer Nano-ZS (Malvern Instruments, Worcestershire, UK). The measurements were performed in 20 mM HEPES buffer (pH 7.4) at room temperature. To test samples' serum stability, the samples were diluted 100 times with PBS supplemented with 10% fetal bovine serum (FBS) and placed in a 37° C. water bath. Hydrodynamic size measurements were made at various time points within 3 weeks.

### Gel Electrophoresis Retardation Assay

Free mRNA, PEI-mRNA, PFHA-PEI-mRNA and PFHA-PEI-mRNA-HP (with varying HP amounts) samples were added to 1% agarose gel at 1 μg mRNA per lane. Gel electrophoresis was run for about 30 min at 120 V. Gels were stained with 0.5 ug/mL ethidium bromide and visualized using a Bio-Rad Universal Hood II Gel Doc System.

### TEM Imaging

TEM samples were prepared by the addition of 4 μL of PEI-mRNA, PFHA-PEI-mRNA or PFHA-PEI-mRNA-HP (with varying HP amounts) solution to a Formvar/carbon coated 300-mesh copper grid (Ted Pella, Inc., Redding, CA) and stained with 1% uranyl acetate and subsequently allowed to air dry. TEM images were acquired on a Tecnai G2 F20 electron microscope (FEI, Hillsboro, OR) operating at a voltage of 200 kV.

### Cell Culture

4T1 and MMC mouse breast cancer cells were cultured in RPMI1640 medium supplemented with 10% vol/vol FBS and 1% vol/vol antibiotic-antimycotic. MCF7 human breast cancer cells, HepG2 human liver cancer cells, SF763 human glioblastoma cells, and C6 rat glioma cells were cultured in DMEM medium supplemented with 10% vol/vol FBS and 1% vol/vol antibiotic-antimycotic. Culture media were replenished once every three days if cells are not confluent enough to be passaged. When cell density reached 80%, 4T1, MCF7, HepG2, SF763, and C6 cells were dissociated with TrypLE agent, MMC with PBS+2.5% v/v EDTA. Dissociated cells were suspended in their corresponding culture media and pelleted at 500 G for 5 mins. Desired numbers of cells were then transferred to new culture flasks with fresh culture media. Cultures were maintained in a 37° C. and 5% CO2 humidified incubator.

### Cellular Uptake and Endosomal Escape Studies

mRNA was labeled with Cy5 following the manufacturer's protocol of the Label IT Tracker Intracellular Nucleic Acid Labeling Kit before complexed into PEI-mRNA, PFHA-PEI-mRNA, and PFHA-PEI-mRNA-HP. 4T1, MMC, and HepG2 cells were seeded at 15,000 cells per well in 8-well glass chambers. All cells were incubated for 24 hours before treatments were added. PEI-mRNA, PFHA-PEI-mRNA, and PFHA-PEI-mRNA-HP were then added to cells at 2 μg/mL mRNA concentration, incubated for either 2 hours or 12 hours before adding 75 nM of Lysotracker Red DND reagent, and then incubated for another 1 hour. There were two identical sets of samples for the 12-hour time point experiment, among which one set was incubated normally in 37° C. incubator while another set was incubated in refrigerator at 4° C. The refrigerated sample was briefly placed at room temperature for adding Lysotracker reagent and was immediately returned to 4° C. for 1 hour incubation. All cells were then washed three times with cold PBS and fixed with paraformaldehyde (4% in PBS) for 15 mins at room temperature. The fixed cells were further washed with cold PBS three times. NucBlue FixCell ReadyProbe DAPI reagent was diluted 10 times in cold PBS and 100 μL was added to each well. Confocal images were acquired using a Leica SP8X confocal laser scanning microscope (Leica, Germany).

### In Vitro Cell Transfection

4T1 and C6 cells were seeded at 4,000 cells per well in 96-well plates. MMC, MCF7, HepG2, and SF763 were seeded at 8,000 cells per well in 96-well plates. All cells were incubated for 24 h after seeded on plates before treatments were added. PEI-mRNA, PFHA-PEI-mRNA, PFHA-PEI-mRNA-HP or Lipofectamine 2000-mRNA complexes were added to 100 μL of fully supplemented culture medium to give a final mRNA concentration of 2 μg/mL in each well for all cancer cell lines. The cells were incubated with complexes for 48 h and the cell culture media were replenished after 24 h. Transfections using the commercial agent, Lipofectamine 2000, were performed following the manufacturer's protocol. The cells were imaged 48 h post-transfection with a Nikon TE300 inverted fluorescent microscope (Nikon, Tokyo, Japan).

### Quantitative Analysis of Transfection Via Flow Cytometry

After cells have been transfected following the in vitro cell transfection procedures, 40 μL TrypLE was added to each well and the wells were incubated for 8 mins to dissociate adherent cells. 100 μL cold PBS was then added to the trypsinized wells to resuspend cells. The cell suspension was collected in 1.5 mL microtubes and centrifuged at 4° C. at 500 G for 5 mins to pellet cells. Cell pellets were then resuspended in 200 μL cold PBS and transferred to flow cytometry tubes for immediate flow cytometry analysis on FACSCanto II (BD Biosciences) from which data was post-processed using FlowJo software (Treestar, Inc., San Carlos, CA).

### In Vitro Cell Viability Studies

4T1, MMC, and HepG2 cells were seeded at 4,000, 8,000, 8,000 cells per well in 96-well plates, respectively. All cells were incubated for 24 h after seeded on plates before treatments were added. The cells were then treated with PEI-mRNA, PFHA-PEI-mRNA, PFHA-PEI-mRNA-HP or Lipofectamine 2000-mRNA at mRNA concentrations of 0, 0.5, 1, 2, and 3 μg/mL. The cells were treated for 24 h before the cell viability was determined using the Alamar Blue assay. The fluorescent signal readout was obtained by a SpectraMax i3 microplate reader (Molecular Devices, Sunnyvale, CA, USA) with 550 nm excitation and 590 nm emission. The fluorescence intensities of all the treatment groups were normalized so that the viability of the untreated cell group was 100%.

Functionality Test after Above 0° C. Storage

PFHA-PEI-mRNA-HP and Lipofectamine 2000-mRNA complexes were prepared on day 0 and were kept in storage at 4° C. throughout this study. 4T1 and HepG2 cells were seeded at 4,000 and 12,000 cells per well in 96-well plates respectively. All cells were incubated for 24 h after seeded on plates before treatments were added. PFHA-PEI-mRNA-HP and Lipofectamine 2000-mRNA complexes were added to 100 μL of fully supplemented culture media to give a final mRNA concentration of 2 μg/mL in each well on day 0, 1, 2, 3, 4, 7, and 15 after sample preparations. The cells were incubated with complexes for 24 h before imaged with a Nikon TE300 inverted fluorescent microscope (Nikon, Tokyo, Japan).

### Stem Cell Transfection

Human induced pluripotent stem cells (WTC-11, GM25256, Coriell Institute, USA) were transferred from Institute for Stem Cell & Regenerative Medicine (University of Washington, WA, USA).

Human induced pluripotent stem cells (hiPSCs) were expanded and cultured according to the Allen Institute protocol for hiPSC lines. Briefly, hiPSCs were cultured on Matrigel-coated tissue culture dishes as a monolayer and supplied with fresh mTeSR1 medium every day. Cells were passaged every 3-4 days and dissociated with accutase in 70-80% confluent. The hiPSCs used in the experiments were at P38-P40. Cultures were maintained in a 37° C. and 5% CO₂ humidified incubator.

hiPSCs were seeded at 10% confluency in 96-well plates. All cells were incubated for 24 h after seeded on plates before treatments were added. PEI-mRNA, PFHA-PEI-mRNA, PFHA-PEI-mRNA-LHP or Lipofectamine 2000-mRNA complexes were added to 100 μL of fully supplemented culture medium to give a final mRNA concentration of 2 μg/mL in each well for all cancer cell lines and 1 μg/mL for hiPSCs. The cells were incubated with complexes for 48 h and the cell culture media were replenished after 24 h. Transfections using the commercial agent, Lipofectamine 2000, were performed following the manufacturer's protocol. The cells were imaged 48 h post-transfection with a Nikon TE300 inverted fluorescent microscope (Nikon, Tokyo, Japan).

In general, the stem cells were cultured, transfected, characterized, evaluated, and tested as described above for the cancer cells.

### Statistical Analysis

The results are presented as mean values±standard error of the mean. The statistical differences were determined by two-sided unpaired Student's t-test. The values were considered statistically significant at p<0.05. In figure presentation, n.s. means statistically not significant, * means p<0.05, ** means p<0.01, *** means p<0.001.

While illustrative embodiments have been illustrated and described, it will be appreciated that various changes can be made therein without departing from the spirit and scope of the disclosure.

1. A nanoparticle for delivery of mRNA to cell, comprising:
(a) a core comprising a polyethylenimine polymer having fluorinated groups covalently coupled thereto and with mRNA reversibly associated therewith; and
(b) a shell surrounding the core comprising heparin.

2. The nanoparticle of claim 1 further comprising a targeting agent associated with the shell.

3. The nanoparticle of claim 2, wherein the targeting agent is selected from the group consisting of agents that bind to receptors overexpressed on tumor cells, agents that bind to cell surface antigens that are expressed on pluripotent stem cells, agents that bind to cell surface antigens on T cells, and agents that bind to antigens presented by MHC molecules.

4. The nanoparticle of claim 1, wherein the polyethylenimine polymer to which the fluorinated groups are covalently coupled thereto has a molecular weight from about 0.8 kDa to about 8 kDa.

5. The nanoparticle of claim 1, wherein each fluorinated group has a molecular weight of about 350 g/mole, or each fluorinated group includes from about 11 to about 15 fluoro (—F) groups, or each fluorinated group includes from about 4 to about 6 difluoromethylene (—CF₂—) groups.

6. The nanoparticle of claim 1, wherein the fluorinated group is present in the polymer complex from about 45 to about 60 weight percent based on the total weight of the polymer, or the molar ratio of fluorinated group to PEI polymer to which the fluorinated group is covalently coupled is from about 5 to about 9.

7. The nanoparticle of claim 1, wherein the mRNA comprises from about 20 to about 4000 nucleotides.

8. The nanoparticle of claim 1, wherein the weight ratio of the mRNA to polymer complex is about 15:1, or the weight ratio of mRNA to heparin in the polyethylenimine polymer complex is from about 1:1 to about 1:5.

9. The nanoparticle of claim 1, wherein the heparin has a molecular weight from about 1.0 kDa to about 30 kDa.

10. The nanoparticle of claim 1, wherein the heparin is present in the polymer from about 1 to about 8 weight percent based on the total weight of the polymer, or the molar ratio of the heparin to PEI polymer complex is from about 1:10 to about 1:200.

11-13. (canceled)

14. A pharmaceutical composition comprising the nanoparticle of claim 1 and a pharmaceutically acceptable carrier.

15. A method for making a nanoparticle for delivery of mRNA to cell, comprising:
(a) mixing a solution of mRNA with a polyethylenimine polymer having fluorinated groups covalently coupled thereto to provide a polyethylenimine polymer having fluorinated groups covalently coupled thereto with associated mRNA; and
(b) mixing a solution of heparin with the polyethylenimine polymer having fluorinated groups covalently coupled thereto with associated mRNA to provide a polyethylenimine polymer having fluorinated groups covalently coupled thereto with associated mRNA and heparin.

16-18. (canceled)

19. A method for introducing mRNA into a cell, comprising contacting a cell with a nanoparticle of claim 1.

20. (canceled)

21. A method for treating a cancer, comprising contacting a cancer cell to be treated with a nanoparticle of claim 1, wherein the mRNA of the nanoparticle is effective for treating the cancer.

22. A method for treating a cancer in a subject, comprising administering to a subject in need thereof a therapeutically effective amount of a nanoparticle of claim 1, wherein the mRNA of the nanoparticle is effective for treating the cancer.

23. The method of claim 21, wherein the cell is a cancer cell.

24. The method of claim 21, wherein the cell is a brain cancer cell, a breast cancer cell, a liver cancer cell, an ovarian cancer cell, a prostate cancer cell, a kidney cancer cell, a lymphoma cell, a melanoma cell, a sarcoma cell, a neural cell, a fibroblast, a muscle cell, a cartilage cell, a bone cell, and their corresponding cancer stem cells, or a human induced pluripotent stem cell.

25. (canceled)

26. The method of claim 22, wherein the cancer is a brain cancer, a breast cancer, a liver cancer, an ovarian cancer, a prostate cancer, a kidney cancer, a lymphoma, a melanoma, or a sarcoma.

27. A method for inhibiting cancer cell growth, comprising contacting a cancer cell with a nanoparticle of claim 1 to introduce one or more pre-selected mRNAs into a cell to induce the expression of one or more pre-determined target proteins in the cell thereby inhibiting cancer cell growth or activating immune cells to eradicate cancer cells.

28. A method for modifying the function of a stem cell, comprising contacting a stem cell with a nanoparticle of claim 1 to introduce one or more pre-selected mRNAs into the cell to induce the expression of one or more pre-determined target proteins in the cell thereby modifying the function of the stem cell.

29-32. (canceled)
